(19) United States
(12) Patent Application Publication
(10) Pub. No.: US 2023/0158080 A1
(43) Pub. Date: May 25, 2023

(54) HORMONAL MANIPULATION OF FIBROBLAST THERAPEUTIC ACTIVITY

(71) Applicant: SPINALCYTE, LLC, Houston, TX (US)

(72) Inventors: Pete O'HEERON, Houston, TX (US); Thomas ICHIM, San Diego, CA (US)

(21) Appl. No.: 18/057,262

(22) Filed: Nov. 21, 2022

Related U.S. Application Data
(60) Provisional application No. 63/282,073, filed on Nov. 22, 2021.

(51) Int. Cl. A61K 35/33; A61K 31/566; A61K 31/568; A61K 31/4045

(52) U.S. Cl. CPC A61K 35/33; A61K 31/566; A61K 31/568; A61K 31/4045

(57) ABSTRACT

Disclosed are means of enhancing therapeutic activities of fibroblasts through manipulation of nuclear receptor activation accomplished by, intra alia, alteration in hormone levels. In some embodiments angiogenic, and/or neurogenic, and/or immunomodulatory activities of fibroblasts are increased by normalization or augmentation of sex-specific hormones. In one embodiment a candidate for fibroblast therapy is administered hormones, pro-hormones, or growth factors to augment therapeutic activities of fibroblasts. In some embodiments fibroblasts are treated in vitro prior to administration with various hormones and/or growth factors in order to augment therapeutic activities of said cells.

This application claims priority to U.S. Provisional Application Serial No. 63/282,073, filed Nov. 22, 2021, which is incorporated by reference herein in its entirety.

### BACKGROUND

### I. Field of the Disclosure

This disclosure relates at least to the fields of cellular biology, physiology, and medicine.

### II. Background

The utilization of testosterone for regenerative activities has previously been reported. For example, stimulation of endothelial progenitor cell activity and mobilization has been reported by testosterone [1-3]. In one clinical study, Blood endothelial progenitor cells (EPCs) and endothelial microparticles (EMPs) were relied upon as markers of endothelial dysfunction. Fifty patients (50-64 years) with erectile dysfunction and late onset hypogonadism were selected. EPC (CD45(neg)/CD34(pos)/CD144(pos)) and EMP (CD45(neg)/CD34(neg)/CD144(pos)) blood concentrations were evaluated by flow cytometry. Thirty patients received androgen replacement therapy (Tostrex® ProStrakan) for 6 months (group A), other 20 patients not received androgen therapy for the contraindications in their clinical history (group B). After 6 months, group B showed IIEF-5 score, peak systolic velocity and acceleration time significantly worse than group A; in addition EPCs and EMPs were significantly higher in group B compared to group A. The authors concluded that patients with isolated arterial ED and LOH not treated with androgen therapy showed worst vascular parameters measured by penile Doppler and higher EPCs and EMPs compared to treated hypogonadal patients, hence, LOH appears to be an additional vascular risk factor, and these markers may be considered as predictors of cavernous artery disease. Finally, androgen therapy improves endothelial dysfunction [4].

At a molecular level, it appears that the androgen receptor is directly involved in ability of testosterone to enhance EPC activity. In one study, researchers found that stimulation of these cells with the synthetic androgen methyltrienolone (R1881) caused androgen receptor translocation in the nucleus, suggesting its activation. Colony forming unit (CFU), proliferation and migration assays under different doses erof R1881 demonstrated a dose-dependent increase in EPC proliferation, migration and colony formation. All these effects are abolished by flutamide pretreatment [5].

Lack of testosterone correlates with reduced EPC activity [6-13]. Administration of testosterone has been shown to reverse deficiencies in EPC number and function in patients that are hypogonadal. For example, in one study, 46 men (age range, 40-73 years; mean age, 58.3 years) with hypogonadal symptoms were recruited, and 29 men with serum total testosterone (TT) levels less than 350 ng/dL received testosterone replacement therapy (TRT) using transdermal testosterone gel (Androgel; 1% testosterone at 5 g/day) for 12 months. Circulating EPC numbers (per 100 000 monocytes) were calculated using flow cytometry. There was no significant association between serum TT levels and the number of circulating EPCs before TRT. Compared with the number of mean circulating EPCs at baseline (9.5 ± 6.2), the number was significantly higher after 3 months (16.6 ± 11.1, p = 0.027), 6 months (20.3 ± 15.3, p = 0.006) and 12 months (27.2 ± 15.5, p = 0.017) of TRT. Thus, it was concluded that serum TT levels before TRT are not significantly associated with the number of circulating EPCs in men with late onset hypogonadism. However, TRT can increase the number of circulating EPCs, which implies the benefit of TRT on endothelial function in hypogonadal men [14].

The effect of testosterone on EPC function and numbers has been shown in other systems, for example, in one study, CD34+/F1k1+ and CD34+/VE-cadherin+ cells were detected in the cavemosal sinusoidal endothelial space. This combination of markers is believed to represent the EPC compartment and the cells represent about 3.79% of the corpus cavernosum in normal rats. The percentage of EPC marker-positive cells decreased significantly when animals were castrated and was restored after testosterone supplementation. Confocal microscopy revealed that the numbers of CD34+/F1k1+ and CD34+/VE-cadherin+ cells decreased in castrated rats compared with controls, but were similar to control levels in rats receiving testosterone replacement. These studies support the need for optimized levels of testosterone in order for EPC numbers to be normalized [15]. Numerous papers have supported proliferative and upregulation of function effects of testosterone on EPC [1, 16-19], as well as hematopoietic progenitors [20-31]. Estrogen has also been shown to alter hematopoietic stem cells as well as to protect them from various inhibitors [32-53].

The present disclosure satisfies a need in the art of addressing medical conditions related to hormones, including hormone imbalances.

### BRIEF SUMMARY

Embodiments of the present disclosure concern method for preventing or treating a disease, disorder, syndrome, symptom, and/or condition. The disease, disorder, syndrome, symptom, and/or condition may comprise depression, a wound, blindness, arthritis, ischemia, diabetes, endometriosis, multiple sclerosis, spinal cord injury, stroke, cancer, a lung disease, a blood disease, a neurological disease, an enzyme or hormone deficiency, a metabolic disorder, an autoimmune disease, age-related macular degeneration, retinal dystrophy, an infectious disease, hemophilia, a degenerative disease, an age-related disease, or a combination thereof. In some embodiments, the arthritis is osteoarthritis or rheumatoid arthritis. In some embodiments, the neurological disease Parkinson’s disease, Alzheimer’s disease, Huntington’s disease, or amyotrophic lateral sclerosis. In some embodiments, the metabolic disorder is a lysosomal storage disorder, galactosemia, maple syrup urine disease, phenylketonuria, a glycogen storage disease, a mitochondrial disorder, Friedrich’s ataxia, a peroxisomal disorder, a metal metabolism disorder, or an organic acidemia. In some embodiments, the autoimmune disease is psoriasis, systemic lupus erythematosus, Grave’s disease, inflammatory bowel disease, Addison’s disease, Sjogren’s syndrome, Hashimoto’s thyroiditis, vasculitis, autoimmune hepatitis, alopecia areata, autoimmune pancreatitis, Crohn’s disease, ulcerative colitis, or dermatomyositis. In some embodiments, the degenerative disease is Charcot-Marie-Tooth disease, chronic obstructive pulmonary disease, chronic traumatic encephalopathy, Creutzfeldt-Jakob disease, cystic fibrosis, cytochrome-C oxidase deficiency, Ehlers-Danlos syndrome, essential tremor, fribrodisplasia ossificans progressiva, infantile neuroaxonal dystrophy, keratoconus, keratoglobus, muscular dystrophy, neuronal ceroid lipofuscinosis, a prion disease, progressive supranuclear palsy, sandhoff disease, spinal muscular atrophy, or retinitis pigmentosa. In some embodiments, the age-related disease is atherosclerosis, a cardiovascular disease, cataracts, osteoporosis, or hypertension. In some embodiments, the cardiovascular disease is angina, or a myocardial infarction.

In some embodiments, the method comprises the steps of contacting a population of fibroblasts (including fibroblast cells; fibroblast-like cells; and/or extracellular vesicles including exosomes, microvesicles, apototic bodies or any other fragments or biologic components of fibroblast cells) with one or more hormones or hormone-like substances, thereby generating a contacted population, and administering a therapeutically effective amount of the contacted population to an individual. In some embodiments, the contacting and administering are done substantially simultaneously, although in some embodiments the contacting and administering are done at different times. In certain embodiments, the contacting comprises culturing the fibroblasts (including fibroblast cells; fibroblast-like cells; and/or extracellular vesicles including exosomes, microvesicles, apototic bodies or any other fragments or biologic components of fibroblast cells) in the presence of the one or more hormones or hormone-like substances. The contacting and administering may or may not be done by the same person or persons.

The hormone(s) may be any hormone(s), such as a sex hormone(s). In some embodiments, the hormone comprises testosterone, estrogen, adrenalin, melatonin, noradrenalin, norepinephrine, triiodothyronine (T3), thyroxine (T4), dopamine, prostaglandin-E2, prostaglandin-E1, leukotrienes, prostacyclin, thromboxane, amylin, anti-mullerian hormone, adiponectin, adrenocorticotropic hormone (ACTH), angiotensin, angiotensinogen, antidiuretic hormone (ADH), atrial natriuretic peptide (ANP), B-type natriuretic peptide (BNP), calcitonin, cholecystokinin (CCK), corticotrophin-releasing hormone (CRH), cortistatin, enkalphin, endothelin, estradiol, erythropoietin (EPO), follicle-stimulating hormone (FSH), galanin, gastric inhibitory peptide (GIP), gastrin, leukotriene, leptin, or a combination thereof.

The fibroblast population (including fibroblast cells; fibroblast-like cells; and/or extracellular vesicles including exosomes, microvesicles, apototic bodies or any other fragments or biologic components of fibroblast cells) may be allogeneic, autologous, or xenogeneic with respect to the individual receiving the fibroblast population. In some embodiments, the fibroblast population comprises fibroblasts that are mitotically active. In some embodiments, the fibroblasts are plastic adherent. The fibroblast population may be derived from any tissue. In some embodiments, the fibroblast population is derived from tissue selected from the group consisting of skin, bone marrow, blood, mobilized peripheral blood, gingiva, tonsil, placenta, Wharton’s Jelly, hair follicle, fallopian tube, liver, deciduous tooth, vas deferens, endometrial, menstrual blood, omentum, and a combination thereof.

The fibroblasts (including fibroblast cells; fibroblast-like cells; and/or extracellular vesicles including exosomes, microvesicles, apototic bodies or any other fragments or biologic components of fibroblast cells) may express or comprise CD105 (also called Endoglin). The fibroblast population may be obtained using CD105 as a selection marker. In some embodiments, the fibroblasts are obtained by a purification method for the marker CD 105. The purification method may be any suitable purification method, such as flow cytometric purification and/or magnetic activated cell sorting.

Fibroblasts (including fibroblast cells; fibroblast-like cells; and/or extracellular vesicles including exosomes, microvesicles, apototic bodies or any other fragments or biologic components of fibroblast cells) may be administered to an individual at any concentration useful for achieving the desired outcome. In some embodiments, a fibroblast population comprising 100,000 to 300 million fibroblasts (or any range derivable therein) are administered to an individual.

In some embodiments, the method comprises the step of administering to an individual, such as an individual having or suspected of having endometriosis, depression, and/or a wound, one or more hormones and a population of fibroblasts. The population of fibroblasts may have been contacted with the one or more hormones, including one or more sex hormones. The population of fibroblasts may have been cultured with the one or more hormones, including one or more sex hormones. In some embodiments, a fibroblast population comprising 100,000 to 300 million fibroblasts, 100,000 to 100 million fibroblasts, or 100,000 to 10 million fibroblasts are administered to an individual.

In some embodiments, an individual having or suspected of having endometriosis is administered a population of fibroblasts and estrogen. The estrogen may be administered at a concentration of 10 ng/kg of body weight to 10 mg/kg of body weight, 100 ng/kg of body weight to 10 mg/kg of body weight, or 10 ng/kg of body weight to 1 mg/kg of body weight.

In some embodiments, an individual suffering from a wound is administered a population of fibroblasts and leptin. The fibroblast population and leptin may be administered at a concentration sufficient to accelerate wound healing. The fibroblast population and leptin may be administered at a concentration sufficient to stimulate polarization of M1 macrophages to M2 macrophages. In some embodiments, the leptin is administered at a concentration of 1 pg/kg of body weight to 100 µg/kg of body weight, 1 ng/kg of body weight to 100 µg/kg of body weight, 100 ng/kg of body weight to 100 µg/kg of body weight, 100 ng/kg of body weight to 10 µg/kg of body weight, or 100 ng/kg of body weight to 1 µg/kg of body weight.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### DETAILED DESCRIPTION

### I. Definitions

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed disclosure.

As used herein, the phrase “hormone treated fibroblasts” includes fibroblasts (including fibroblast cells; fibroblast-like cells; and/or extracellular vesicles including exosomes, microvesicles, apototic bodies or any other fragments or biologic components of fibroblast cells) that have been contacted with at least one hormone, including any hormone disclosed herein. The hormone treated fibroblasts may be cultured with at least one hormone, including any hormone disclosed herein. The contact may be for any duration and at any concentration.

The term “undifferentiated” refers to cells that have not become specialized cell types.

A “nutrient medium” is a medium for culturing cells containing nutrients that promote proliferation.

The phrases “pharmaceutically acceptable” or “pharmacologically acceptable” refer to molecular entities and compositions that do not produce an adverse, allergic, or other untoward reaction when administered to an individual. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

In some embodiments, the term “agent” or “therapeutic agent” refers to any cell, fibroblast, fibroblast population, hormone, or other therapeutic composition disclosed herein.

As used herein, the term “sex hormones” (also known as sex steroids, gonadocorticoids and gonadal steroids) as used herein refers to steroid hormones that interact with vertebrate steroid hormone receptors. Sex hormones may include any of androgen, estrogen, and/or progestogen.

It is contemplated that any embodiment discussed in this specification can be implemented with respect to any method or composition of the disclosure, and vice versa. Furthermore, compositions of the disclosure can be used to achieve methods of the disclosure.

### II. General Embodiments

Certain embodiments herein concern methods of enhancing therapeutic activity of a fibroblast population (including fibroblast cells; fibroblast-like cells; and/or extracellular vesicles including exosomes, microvesicles, apototic bodies or any other fragments or biologic components of fibroblast cells), such as compared to the therapeutic activity of the fibroblast population in the absence of the enhancement condition and/or environment. In some embodiments, the method comprises one or more of the steps of selecting a fibroblast population (including fibroblast cells; fibroblast-like cells; and/or extracellular vesicles including exosomes, microvesicles, apototic bodies or any other fragments or biologic components of fibroblast cells); contacting said fibroblast population with one or more hormone or hormone-like substances; optionally assessing the therapeutic activity of the fibroblast population; and optionally altering concentration of the hormone or hormone-like substance(s) in order to optimize therapeutic activity of the fibroblast population. In specific embodiments, the therapeutic activity is the ability to stimulate angiogenesis. In some embodiments, angiogenesis is associated with generation of new blood vessels from pre-existing blood vessels. In some embodiments, angiogenesis is associated with induction of MMP-3, MMP-7, MMP-9, MMP-12, interleukin-1, interleukin-6, interleukin-10, interleukin-11, interleukin-13, interleukin-17, interleukin-20, interleukin-25, interleukin-35, TNF-alpha, VEGF, IGF-1, EGF-1, HGF-1, FGF-1, FGF-2, and/or FGF-5.

In some embodiments, the therapeutic activity comprises stimulation of progenitor cell proliferation. The progenitor cell may be a myeloid progenitor cell, a myeloid suppressor cell, a hematopoietic stem cell, or a mixture thereof. In some embodiments, the hematopoietic stem cell is capable of reconstituting one or more cell lineages, such as an erythrocytic lineage, a lymphocytic lineage, a megakaryocytic lineage, a myelocytic lineage, or a basophilic lineage. In some embodiments, the hematopoietic stem cell expresses CD34, CD33, CD133, c-met, flt-3 receptor, stem cell factor receptor, interleukin-1 receptor, interleukin-3 receptor, interleukin-8 receptor, interleukin-11 receptor, FGF-1 receptor, FGF-2 receptor, FGF-5 receptor, interferon alpha receptor, interferon gamma receptor, TGF-beta receptor, VEGF receptor, PDGF receptor, IGF-1 receptor, and/or angiopoietin receptor. In some embodiments, the progenitor cell is a myogenic progenitor. The myogenic progenitor may be capable of generating smooth muscle and/or striated muscle. In some embodiments, the myogenic progenitor expresses stem cell factor receptor, c-met, pim-1, oct-4, NANOG, c-myc, and/or KLF-1. In some embodiments, the progenitor cell is a neurogenic progenitor. The neurogenic progenitor may be found in the hippocampus, including the dentate gyrus of the hippocampus, or the subventricular zone. In some embodiments, the neurogenic progenitor lacks expression of dopamine receptor 2. In some embodiments, the neurogenic progenitor expresses trk-1, BDNF receptor, steel factor receptor, G-CSF receptor, M-CSF receptor, GM-CSF receptor, LIF receptor, dopamine receptor 1, TNF-alpha receptor p55, TNF-alpha receptor p75, neurogenin, gp120, GDNF receptor, CTNF receptor, and/or serotonin receptor. In some embodiments, the neurogenic progenitor is capable of differentiating into spiny neurons.

In some embodiments, the progenitor cell is a cardiogenic progenitor cell. The cardiogenic progenitor cell may expresses CDX-1, PIM-1, troponin, aldehyde dehydrogenase, a molecule capable of effluxing rhodamine 231, isl-1, SOX-2, and/or KLF4. In some embodiments, the progenitor cell is a renal progenitor cell. The renal progenitor cell may express stem cell factor receptor.

In some embodiments, fibroblasts are either allogeneic, autologous, or xenogeneic with respect to the individual receiving the fibroblasts (including fibroblast cells; fibroblast-like cells; and/or extracellular vesicles including exosomes, microvesicles, apototic bodies or any other fragments or biologic components of fibroblast cells). In some embodiments, the fibroblasts are mitotically active prior to administration into a recipient in need of treatment.

In some embodiments, fibroblasts are isolated from a tissue such as skin, bone marrow, blood, mobilized peripheral blood, gingiva, tonsil, placenta, Wharton’s Jelly, hair follicle, fallopian tube, liver, deciduous tooth, vas deferens, endometrial, menstrual blood, omentum, a combination thereof, or any other region of the body which may produce acceptable fibroblasts. In some embodiments, the fibroblasts are obtained by plastic adherence. In some embodiments, the fibroblasts are obtained by flow cytometric purification for the marker CD-105 and/or magnetic activated sorting (MACS) purification for the marker CD 105.

In some embodiments, fibroblasts are cultured with a hormone including, for example, testosterone, adrenalin, melatonin, norepinephrine, dopamine, T3,T4, amylin, adiponectin, angiotensin, antidiurentic hormone, calcitonin, estrogen, PGE-1, PGE-1, leukotriene, enkalphin, endothelin, or a combination thereof.

Certain embodiments concern methods of treating rheumatoid arthritis by administering a therapeutically effective amount of one or more hormones or hormone-like substances at an appropriate concentration and/or frequency to reduce pathology of the rheumatoid arthritis. In some embodiments, the pathology is formation of a pannus. The pannus may be associated with matrix metalloprotease activation. The matrix metalloprotease includes MMP-1, MMP-3, and/or MMP-12. In some embodiments, the pannus comprises activated fibroblasts. The activated fibroblasts may be endogenous to the pannus and may be a result of the pathology. Activated fibroblasts may possess gelatinase activity, neutrophil recruiting activity, monocyte recruiting activity, and/or T cell recruiting activity. The T cells being recruited may be CD4 T cells, including Th1 cells. The Th1 cells may express STAT1, STAT4, T-bet, CCR1, CCR5, CXCR3, CD119, interferon gamma receptor 2, interleukin-10 receptor, CD25, interleukin-12 receptor alpha, interleukin-12 receptor beta, interleukin-18 receptor alpha, interleukin-18 receptor beta, interleukin-27 receptor alpha, interleukin-27 receptor beta, and/or interleukin-33 receptor alpha. In some embodiments, the Th1 cell secretes interleukin-1, interleukin-2, interleukin-7, interleukin-8, interleukin-12, interleukin-15, interleukin-16, interleukin-18, interleukin-33, TNF-alpha, TNF-beta, and/or interferon gamma. In some embodiments, the CD4 cells are Th17 cells. The Th17 T cells may be capable of stimulating production of TNF-alpha from naive T cells and/or myeloid lineage cells. The myeloid lineage cells may be myeloid derived suppressor cells. In some embodiments, the myeloid lineage cells are monocytes, Kupffer cells, scar-associated macrophage cells, lipid-associated macrophage cells, and/or glial cells. In some embodiments, the Th17 T cell is capable of stimulating production of TNF-alpha from endothelial cells and/or pulmonary epithelial cells.

In some embodiments, the Th17 T cells produce interleukin-17A, interleukin-17F, CCL20, interleukin-21, interleukin-22, and/or interleukin-26. In some embodiments, the Th17 T cells express BatF, IRF4, ROR-alpha, ROR-gamma,s STAT3, CCR4, CCR6, IL-1 receptor alpha, IL-1 receptor beta, IL-6 receptor alpha, IL-6 receptor beta, IL-21 receptor, IL-23 receptor, and/or TGF-beta receptor II.

Certain embodiments concern methods of protecting against and/or treating endometriosis, including by one or more of the steps of optionally selecting an individual in need of treatment; administering to an individual in need of treatment one or more hormones or hormone-like substances in a manner to alter fibroblast activity; and optionally adjusting said intervention based on therapeutic activity accomplished. The hormone may interact with the fibroblasts (including fibroblast cells; fibroblast-like cells; and/or extracellular vesicles including exosomes, microvesicles, apototic bodies or any other fragments or biologic components of fibroblast cells) to induce upregulation of bcl-2, bcl-2x, FGF-1 receptor, FGF-2 receptor, and/or FGF-5 receptor in a cell, such as the fibroblasts. In some embodiments, the fibroblasts are autologous, allogeneic, or xenogeneic with respect to an individual, including the individual in need of treatment. In some embodiments, the fibroblasts are treated with estradiol before administration.

Certain embodiments concern methods of treating depression including by administrating to an individual that has depression or is at risk for depression (e.g. family or personal history) a therapeutically effective amount of estrogen and/or fibroblasts at a concentration sufficient to treat depression. In some embodiments, the estrogen is administered at a concentration of 10 ng-10 mg per kg of body weight per day, 100 ng-10 mg per kg of body weight per day, or 10 ng-1 mg per kg of body weight per day. In some embodiments, 100,000 to 300 million, 100,000 to 100 million fibroblasts, or 100,000 to 10 million are administered daily.

Certain embodiments concern methods of accelerating wound healing including by providing leptin together with fibroblasts to an individual in need of treatment. In some embodiments, leptin is administered at a concentration sufficient to stimulate polarization of M1 cells to M2. In some embodiments, leptin is administered at 1 pg-100 µg per kg of body weight, 100 ng-100 µg per kg of body weight, 100 ng-10 µg per kg of body weight, or 100 ng-1 µg per kg of body weight.

Certain embodiments concern methods of increasing insulin sensitivity and/or inducing weight loss in an individual including by administering leptin and/or fibroblasts to the individual. In some embodiments, leptin is administered at a concentration of 10 ng-10 µg per kg of body weight per day, 100 ng-10 µg per kg of body weight per day, or 10 ng-1 µg per kg of body weight per day. In some embodiments, 100,000 to 300 million, 100,000 to 100 million fibroblasts, or 100,000 to 10 million are administered daily.

Certain embodiments concern methods of modulating an immune response in an individual including by administering a fibroblast population that has been contacted with one or more hormones to the individual. The hormone may be any hormone including, for example, testosterone, estrogen, adrenalin, melatonin, noradrenalin, norepinephrine, triiodothyronine (T3), thyroxine (T4), dopamine, prostaglandin-E2, prostaglandin-E1, leukotrienes, prostacyclin, thromboxane, amylin, anti-mullerian hormone, adiponectin, adrenocorticotropic hormone (ACTH), angiotensin, angiotensinogen, antidiuretic hormone (ADH), atrial natriuretic peptide (ANP), B-type natriuretic peptide (BNP), calcitonin, cholecystokinin (CCK), corticotrophin-releasing hormone (CRH), cortistatin, enkalphin, endothelin, estradiol, erythropoietin (EPO), follicle-stimulating hormone (FSH), galanin, gastric inhibitory peptide (GIP), gastrin, leukotriene, and/or leptin. The fibroblasts may be administered intrathecally, or intervenously, or supcutaneously, or intraperitopeally. or intramuscular, or intradermally.

Certain embodiments concern methods for regulating the endometrium including by administering fibroblasts with one or more hormones. The hormone may comprise any hormone described herein. The fibroblasts may comprise any fibroblasts described herein.

### III. Hormones and Fibroblasts

Embodiments herein provide the novel and unexpected activity of hormones, including testosterone, to augment fibroblast activity. For utilization of frequency and doses, one of skill in the art is referred to the publications encompassed herein demonstrating testosterone can augment various compartments of the body associated with regeneration. In some embodiments, the administration of testosterone is administered to an individual prior to, and/or concurrent with, and/or subsequent to fibroblast therapy. In some embodiments, the individual is a male.

Embodiments also provide the previously unexpected ability of various hormones to alter the physiological properties of fibroblasts including ability to reduce production of inflammatory cytokines in chronic conditions without inducing systemic immune suppression.

In some embodiments of the disclosure, hormones are used to enhance ability of fibroblasts to support differentiation of pancreatic islet progenitors. In some embodiments hormones are selected from a group comprised of factors (such as ilotropin [54], growth hormone [55], insulin [56], prolactin [55, 57], exendin-4 [58-60], GLP-1 [61-65], dapagliflozin [66], Betacellulin [67-69], activin A [70], gastrin [71], EGF [72], IGF-1 [73, 74], IDX-1 [75], reg protein [76-78], neurogenin-3 [79-82], Nidogen-1 [83], HNF-6 [84], SEPT7b [85], SOX-9 [86], heparan sulfate [87], estrogen [88], INGAP [89, 90], ghrelin [91], SDF-1 [92], PDX-1 [91, 93], MAFA [91], and thyrotrophin releasing hormone [94]).

In some embodiments, hormonal manipulation is utilized to inhibit the neoplasiapromoting activity of cancer-associated fibroblasts. For example, in the area of prostate cancer, ablation of testosterone, either by direct inhibition of androgen receptor, or by administering hormones which inhibit testosterone synthesis is utilized as a means of reducing activity of cancer associated fibroblasts.

In one embodiment, flutamide is utilized treatment of a prostate cancer patient as a means of weakening localized immune suppression by targeting the bidirectional interaction between the cancer tissue itself and the surrounding fibroblasts. It is known that cancer associated fibroblasts possess immune suppressive activity in prostate cancer [95]. In one embodiment of the disclosure, hormonal manipulation of a prostate cancer patient is utilized to abrogate the immune suppressive properties of the prostate cancer associated fibroblasts.

In another embodiment, suppression of cancer-associated fibroblast growth factor production is accomplished by hormonal treatments. In one embodiment, depletion of testosterone is utilized to reduce production of GDF-15 from cancer associated fibroblasts. GDF-15 has been heavily implicated in progression, growth and metastasis of cancer [96-128].

In some embodiments, hormonal manipulation of fibroblasts to reduce production of GDF-15 is utilized as a means of potentiating immunotherapy of cancer. The immunotherapy may be antigen specific or antigen non-specific. Studies of adoptive T cell immunotherapy [133] along with recently reported positive clinical results in non-Hodgkins lymphoma [134, 135] and prostate cancer immunotherapy targeting tumor-associated antigens (TAAs) have provided proof of concept that the immune system can support a clinically effective anti-tumor immune response [132]. Although the benefits of anti-tumor immunotherapy has not been demonstrated in a wide range of tumor types, it has been postulated that the missing critical element is a sufficiently potent, readily translatable cancer vaccine strategy [136]. Patients with breast cancer can have endogenous or immunotherapy elicited humoral and cellular responses to several tumor-associated antigens (TAAs) [137-146], however, these have generally been of sub-optimal magnitude with elusive clinical efficacy. Additionally, breast cancer patients with significant inflammatory infiltrates, i.e. medullary breast carcinoma, have significantly improved survival despite greater cellular anaplasia[147-149]. Thus, it is reasonable to hypothesize that a sufficiently potent, antigen-specific immunotherapeutic strategy for breast cancer would have clinical efficacy and offer a valuable new treatment modality.

A variety of TAAs have been identified in breast cancer consisting of overexpressed normal proteins and mutated proteins that are normally found in breast tissue, however, only a minority of the TAAs that have been discovered so far are immunogenic, which limits the potential use for immunotherapy. In addition, while the overwhelming majority of TAAs are expressed in tumor cells, they are typically also expressed in a variety of normal cells, e.g. the breast cancer TAAs; epidermal growth factor receptors (HER2), carcinoembryonic antigen (CEA), mucin (MUC1), the tumor suppressor protein p53, and telomerase reverse transcriptase (TERT). Thus, they are recognized by the immune system as self-molecules, and the immune system has protective mechanisms for preventing recognition of self-tissue antigens and autoimmune responses. Additionally, tumors employ other mechanisms for escaping immune surveillance, such as: (i) low level expression of MHC class I molecules [150]; (ii) lack of expression of B7 (CD80/CD86) co-stimulatory molecules [151]; (iii) production of cytokines that stimulate the accumulation of immune-suppressor cells [152, 153]; and (iv) ineffective processing and presentation of self-antigens by “professional” antigen-presenting cells (APC) [154]. This probably explains why TAA or tumor cell vaccines that have been used in clinical trials generally do not induce strong protective immunity [133]. Identification of novel TAA that are not expressed on normal cells may provide an attractive alternative particularly if combined with potent immunotherapeutic platforms, because these antigens are less likely to be subject to the tolerogenic mechanisms that limit immune responses to “self” antigens and therefore, may be better immunogens.

In one specific embodiment administration of fibroblasts (including fibroblast cells; fibroblast-like cells; and/or extracellular vesicles including exosomes, microvesicles, apototic bodies or any other fragments or biologic components of fibroblast cells) are manipulated by treatment with various hormones. Useful hormones for manipulation of fibroblast activity include testosterone and leptin when enhancement of healing such as wound healing is desired. In some situations, testosterone and/or leptin is added to enhance viability of fibroblasts subsequent to their administration.

In embodiments herein, various hormones may be utilized. Useful hormones for any embodiments herein include testosterone, estrogen, adrenalin, melatonin, noradrenalin, norepinephrine, triiodothyronine (T3), thyroxine (T4), dopamine, prostaglandin-E2, prostaglandin-E1, leukotrienes, prostacyclin, thromboxane, amylin, anti-mullerian hormone, adiponectin, adrenocorticotropic hormone (ACTH), angiotensin, angiotensinogen, antidiuretic hormone (ADH), atrial natriuretic peptide (ANP), B-type natriuretic peptide (BNP), calcitonin, cholecystokinin (CCK), corticotrophin-releasing hormone (CRH), cortistatin, enkalphin, endothelin, estradiol, erythropoietin (EPO), follicle-stimulating hormone (FSH), galanin, gastric inhibitory peptide (GIP), gastrin, leukotriene, and leptin.

In some embodiments of the disclosure, hormonal pretreatment of fibroblasts is utilized to enhance therapeutic activity of said cells in a wide variety of degenerative states. In one embodiment said fibroblasts are utilized to treat diseases selected from a group comprising of blindness, arthritis (e.g., osteoarthritis or rheumatoid arthritis), ischemia, diabetes (e.g., Type 1 or Type 2 diabetes), multiple sclerosis, spinal cord injury, stroke, cancer, a lung disease, a blood disease, a neurological disease, such as Parkinson’s disease, Alzheimer’s disease, Huntington’s disease, and ALS, an enzyme or hormone deficiency, a metabolic disorder (e.g., a lysosomal storage disorder, Galactosemia, Maple syrup urine disease, Phenylketonuria, a glycogen storage disease, a mitochondrial disorder, Friedrich’s ataxia, a peroxisomal disorder, a metal metabolism disorder, or an organic acidemia), an autoimmune disease (e.g., Psoriasis, Systemic Lupus Erythematosus, Grave’s disease, Inflammatory Bowel Disease, Addison’s Diseases, Sjogren’s Syndrome, Hashimoto’s Thyroiditis, Vasculitis, Autoimmune Hepatitis, Alopecia Areata, Autoimmune pancreatitis, Crohn’s Disease, Ulcerative colitis, Dermatomyositis), age-related macular degeneration, retinal dystrophy, an infectious disease, hemophilia, a degenerative disease (e.g., Charcot-Marie-Tooth disease, chronic obstructive pulmonary disease, chronic traumatic encephalopathy, Creutzfeldt-Jakob disease, Cystic Fibrosis, Cytochrome C Oxidase deficiency, Ehlers-Danlos syndrome, essential tremor, Fribrodisplasia Ossificans Progressiva, infantile neuroaxonal dystrophy, keratoconus, keratoglobus, muscular dystrophy, neuronal ceroid lipofuscinosis, a prior disease, progressive supranuclear palsy, sandhoff disease, spinal muscular atrophy, retinitis pigmentosa), or an age-related disease (e.g., atherosclerosis, cardiovascular disease (e.g., angina, myocardial infarction), cataracts, osteoporosis, or hypertension).

For the practice of certain embodiments of the disclosure, fibroblasts (including fibroblast cells; fibroblast-like cells; and/or extracellular vesicles including exosomes, microvesicles, apototic bodies or any other fragments or biologic components of fibroblast cells) may be derived from a variety of tissues. In one embodiment, isolated cells express very little or no SSEA-1 marker. The useful cells of the disclosure also may express high levels of the cell surface antigens that are normally found on hormone treated fibroblasts, but not normally on human stem cells, including CD56, aminopeptidase N, CD44, hyaluronic acid-binding receptor, CD49b, collagen/laminin-binding integrin alpha2, and CD105 (endoglin). In some embodiments, at least 90%, 91%, 92%, 93%, 94%, 95%, 96%, 97%, 98%, 99% of hormone-treated fibroblasts express at least one of the cell surface antigens.

In some embodiments of the disclosure, at least about 90%, 91%, 92%, 93%, 94%, 95%, 96%, 97%, 98%, 99%, or 100% of the cells, such as fibroblasts, express CD56. In additional embodiments, at least about 90%, 91%, 92%, 93%, 94%, 95%, 96%, 97%, 98%, 99%, or 100% of the cells, such as fibroblasts, express CD37. In some embodiments of the disclosure, at least about 90%, 91%, 92%, 93%, 94%, 95%, 96%, 97%, 98%, 99%, 99.5%, or 100% of the cells, such as fibroblasts, express CD127. In further embodiments of the disclosure, at least about 90%, 91%, 92%, 93%, 94%, 95%, 96%, 97%, 98%, 99%, 99.5%, or 100% of the cells, such as fibroblasts, express CD127.

In particular embodiments of the disclosure, the cells are fibroblasts that can be propagated for an indefinite period of time in continuous culture in an undifferentiated state. In some embodiments of the disclosure, hormones are added to the culture when the cells are in an undifferentiated state.

In some embodiments, the hormone vasoactive intestinal peptide is utilized to enhance therapeutic activity of fibroblasts. Utilization of the hormone to manipulate fibroblasts can be based on concentrations and activities described in the literature for other regenerative cells. Publications that are useful to guide one of skill in the art are provided and incorporated herein by reference [155-160].

Fibroblasts that have been exposed to hormones may possess various physiological and/or immunological alterations. In some embodiments, fibroblast are altered to enhance their ability to induce immunological tolerance. Enhancement of tolerogenic properties may be performed in any manner. In some embodiments, one or more (e.g., one, two, three, four, five, six, seven, or all eight) of PD-L1, HLA-G (H2-M3), Cd47, Cd200, FASLG (FasL), Cc121 (Cc121b), Mfge8, and Serpin B9 (Spi6) is expressed in a hormone treated fibroblast at a level that is equal to or greater than the expression level of the corresponding endogenous gene in an untreated fibroblast (e.g., the expression level of the protein in hormone treated fibroblasts is equal to the level of expression of the endogenous gene in untreated fibroblasts, or is 1.5, 2, 3, 4, 5, 6, 7, 8, 9, 10-fold or more higher than the level of expression of the endogenous gene in untreated fibroblasts). In some embodiments, all eight of PD-L1, HLA-G (H2-M3), Cd47, Cd200, FASLG (FasL), Cc121 (Cc121b), Mfge8, and Serpin B9 (Spi6) are expressed at a level that is greater (e.g., 2, 3, 4, 5, 6, 7, 8, 9, 10, 20, 30, 40, 50, or 100-fold higher or more) than the expression level of the endogenous gene in an untreated fibroblast.

In some embodiments, one or more (e.g., one, two, three, four, five, six, seven, or all eight) of PD-L1, HLA-G (H2-M3), Cd47, Cd200, FASLG (FasL), Cc121 (Cc121b), Mfge8, and Serpin B9 (Spi6) is expressed at a level that is in the top 5% of gene expression for all genes in the fibroblast cell genome. In some embodiments, one or more (e.g., one, two, three, four, five, six, seven, or all eight) of PD-L1, HLA-G (H2-M3), Cd47, Cd200, FASLG (FasL), Cc121 (Cc121b), Mfge8, and Serpin B9 (Spi6) is expressed at a level that is in the top 1% of gene expression for all genes in the fibroblast cell genome. In some embodiments, all of PD-L1, HLA-G (H2-M3), Cd47, Cd200, FASLG (FasL), Cc121 (Cc121b), Mfge8, and Serpin B9 (Spi6) are expressed at a level that is in the top 5% of gene expression for all genes in the fibroblast cell genome.

In some embodiments, hormones are administered or are used to manipulate fibroblasts for the treatment of rheumatoid arthritis. In one embodiment, hormones, such as testosterone or inducers of testosterone, are administered with the notion of targeting pannus formation. In other embodiments, hormones, such as vasoactive intestinal protein, are administered for the purpose of inducing immune modulation in a patient suffering from rheumatoid arthritis.

In other embodiments, hormones such as estrogen are utilized to alter fibroblasts in order to treat endometriosis. Various combinations of hormones and fibroblasts are envisioned in the disclosure. For example, hormones such as estradiol may be administered exogenously together with administration of fibroblasts.

For generation of fibroblasts a variety of tissues may be utilized including bone marrow, mobilized peripheral blood, peripheral blood, skin, adipose, umbilical cord, cord blood, and endometrial fibroblasts. In some embodiments, fibroblast populations are derived from one or more tissue including skin, bone marrow, blood, mobilized peripheral blood, gingiva, tonsil, placenta, Wharton’s Jelly, hair follicle, fallopian tube, liver, deciduous tooth, vas deferens, endometrial, menstrual blood, and omentum.

Expansion of fibroblasts may be performed prior to contact with hormones. Means of expanding fibroblasts are known in the art. In certain embodiments, fibroblasts are activated prior to therapeutic use and/or administration of agents which act as “regenerative adjuvants” for said fibroblasts. The cells used in administrations herein may display typical fibroblast morphologies when growing in cultured monolayers. Specifically, cells may display an elongated, fusiform or spindle appearance with slender extensions, or cells may appear as larger, flattened stellate cells which may have cytoplasmic leading edges. A mixture of these morphologies may also be observed. The cells may express proteins characteristic of normal fibroblasts including the fibroblast-specific marker, CD90 (Thy-1), a 35 kDa cell-surface glycoprotein, and the extracellular matrix protein, collagen. In one embodiment, the fibroblasts of the disclosure can also be used to create other cell types for tissue repair or regeneration.

In another embodiment, the hormone-treated fibroblasts (including fibroblast cells; fibroblast-like cells; and/or extracellular vesicles including exosomes, microvesicles, apototic bodies or any other fragments or biologic components of fibroblast cells) may be administered to an individual such that the hormone treated fibroblasts contact microglia and/or astrocytes in the brain to reduce inflammation, whereby the hormone treated fibroblasts limit neurodegeneration caused by activated glial cells in diseases, or disorders such as Alzheimer’s Disease, Parkinson’s Disease, stroke, or brain cell injuries. In yet another embodiment, the hormone treated fibroblasts may be administered to an individual such that the hormone treated fibroblasts contact keratinocytes and Langerhans cells in the epidermis of the skin to reduce inflammation as may occur in psoriasis, chronic dermatitis, and contact dermatitis. Although this embodiment is not to be limited to any theoretical reasoning, it is believed that the hormone treated fibroblasts may contact the keratinocytes and Langerhans cells in the epidermis, and alter the expression of T-cell receptors and cytokine secretion profiles, leading to decreased expression of tumor necrosis factoralpha (TNF-α) and increased regulatory T-cell (T_{reg} cell) population.

In a further embodiment, the hormone-treated fibroblasts (including fibroblast cells; fibroblast-like cells; and/or extracellular vesicles including exosomes, microvesicles, apototic bodies or any other fragments or biologic components of fibroblast cells) may be used to reduce inflammation in the bone, as occurs in arthritis and arthritis-like conditions, including but not limited to, osteoarthritis and rheumatoid arthritis, and other arthritic diseases. Although the scope of this embodiment is not intended to be limited to any theoretical reasoning, it is believed that the hormone treated fibroblasts may inhibit Interleukin-17 secretion by memory T-cells in the synovial fluid. In another embodiment, the hormone treated fibroblasts may be used to limit inflammation in the gut and liver during Inflammatory bowel disease and chronic hepatitis, respectively. Although the scope of this aspect of the present disclosure is not intended to be limited to any theoretical reasoning, it is believed that the hormone-treated fibroblasts promote increased secretion of Interleukin-10 (IL-10) and the generation of regulatory T-cells (T_{reg} cells). In another embodiment, the hormone treated fibroblasts may be used to inhibit excessive neutrophil and macrophage activation in pathological conditions such as sepsis and trauma, including burn injury, surgery, and transplants. Although the scope of this embodiment is not to be limited to any theoretical reasoning, it is believed the hormone-treated fibroblasts promote secretion of suppressive cytokines such as IL-10, and inhibit macrophage migration inhibitory factor.

In another embodiment, the hormone-treated fibroblasts (including fibroblast cells; fibroblast-like cells; and/or extracellular vesicles including exosomes, microvesicles, apototic bodies or any other fragments or biologic components of fibroblast cells) may be used to control inflammation in immune privileged sites such as the eye, including the cornea, lens, pigment epithelium, and retina, brain, spinal cord, pregnant uterus and placenta, ovary, testes, adrenal cortex, liver, and hair follicles. Although the scope of this embodiment is not to be limited to any theoretical reasoning, it is believed that the hormone-treated fibroblasts (including fibroblast cells; fibroblast-like cells; and/or extracellular vesicles including exosomes, microvesicles, apototic bodies or any other fragments or biologic components of fibroblast cells) promote the secretion of suppressive cytokines such as IL-10 and the generation of T_{reg} cells. In yet another embodiment, the hormone treated fibroblasts may be used to treat tissue damage associated with end-stage renal disease (ESRD) infections during dialysis and/or glomerulonephritis. Although the scope of this embodiment is not to be limited to any theoretical reasoning, it is believed that hormone-treated fibroblasts may promote renal repair. Hormone-treated fibroblasts (including fibroblast cells; fibroblast-like cells; and/or extracellular vesicles including exosomes, microvesicles, apototic bodies or any other fragments or biologic components of fibroblast cells) also express and secrete vascular endothelial growth factor, or VEGF, which stimulates new blood vessel formation, which should aid in the repair of damaged kidney tissue.

In a further embodiment, the hormone-treated fibroblasts (including fibroblast cells; fibroblast-like cells; and/or extracellular vesicles including exosomes, microvesicles, apototic bodies or any other fragments or biologic components of fibroblast cells) may be used to control viral infections such as influenza, hepatitis C, Herpes Simplex Virus, vaccinia virus infections, and Epstein-Barr virus. Although the scope of this embodiment is not to be limited to any theoretical reasoning, it is believed that the hormone treated fibroblasts promote the secretion of Interferon-Beta (IFN-β). In yet another embodiment, the hormone treated fibroblasts may be used to control parasitic infections such as Leishmania infections and Helicobacter infections. Although the scope of this embodiment is not to be limited to any theoretical reasoning, it is believed that the hormone treated fibroblasts mediate responses by T helper 2 (Th2) cells, and thereby promote increased production of Immunoglobulin E (IgE) by β-cells.

It is to be understood, however, that the scope of this aspect of the present disclosure is not to be limited to the treatment of any particular inflammatory response. The hormone-treated fibroblasts (including fibroblast cells; fibroblast-like cells; and/or extracellular vesicles including exosomes, microvesicles, apototic bodies or any other fragments or biologic components of fibroblast cells) may be administered to a mammal, including human and non-human primates, as hereinabove described.

The hormone-treated fibroblasts (including fibroblast cells; fibroblast-like cells; and/or extracellular vesicles including exosomes, microvesicles, apototic bodies or any other fragments or biologic components of fibroblast cells) may be administered in conjunction with an acceptable pharmaceutical carrier, as hereinabove described. In accordance with another aspect of the present disclosure, there is provided a method of treating inflammation and/or repairing epithelial damage in an individual. The method comprises administering to the individual hormone treated fibroblasts in an amount effective to treat the inflammation and/or epithelial damage in the individual.

Although the scope of this aspect of the present disclosure is not to be limited to any theoretical reasoning, it is believed that the hormone-treated fibroblasts (including fibroblast cells; fibroblast-like cells; and/or extracellular vesicles including exosomes, microvesicles, apototic bodies or any other fragments or biologic components of fibroblast cells) cause a decrease in the secretion of the pro-inflammatory cytokines TNF-α and Interferon-y by T-cells, and an increase in the secretion of the anti-inflammatory cytokines Interleukin-10 (IL-10) and Interleukin-4 (IL-4) by T-cells. It is also believed that the hormone treated fibroblasts cause a decrease in Interferon-y secretion by natural killer (NK) cells. The inflammation and/or epithelial damage that may be treated in accordance with this aspect of the present disclosure includes, but is not limited to, inflammation and/or epithelial damage caused by a variety of diseases and disorders, including, but not limited to, autoimmune disease, rejection of transplanted organs, burns, cuts, lacerations, and ulcerations, including skin ulcerations and diabetic ulcerations. In one embodiment, the hormone treated fibroblasts (including fibroblast cells; fibroblast-like cells; and/or extracellular vesicles including exosomes, microvesicles, apototic bodies or any other fragments or biologic components of fibroblast cells) are administered to an individual in order to repair epithelial damage resulting from autoimmune diseases, including, but not limited to, rheumatoid arthritis, Crohn’s Disease, Type 1 diabetes, multiple sclerosis, scleroderma, Graves’ Disease, lupus, inflammatory bowel disease, autoimmune gastritis (AIG), and autoimmune glomerular disease. The hormone treated fibroblasts also may repair epithelial damage resulting from graft-versus-host disease (GVHD). This aspect of the present disclosure is applicable particularly to the repair of epithelial damage resulting from graft-versus-host disease, and more particularly, to the repair of epithelial damage resulting from severe graft-versus-host disease, including Grades III and IV graft-versus-host disease affecting the skin and/or the gastrointestinal system. Applicants have discovered, in particular, that hormone-treated fibroblasts (including fibroblast cells; fibroblast-like cells; and/or extracellular vesicles including exosomes, microvesicles, apototic bodies or any other fragments or biologic components of fibroblast cells), when administered to a patient suffering from severe graft-versus-host disease, and in particular, Grades III and IV gastrointestinal graft-versus-host disease, the administration of the hormone treated fibroblasts resulted in repair of skin and/or ulcerated intestinal epithelial tissue in the patient.

In another embodiment, the hormone-treated fibroblasts (including fibroblast cells; fibroblast-like cells; and/or extracellular vesicles including exosomes, microvesicles, apototic bodies or any other fragments or biologic components of fibroblast cells) are administered to an individual in order to repair epithelial damage to a transplanted organ or tissue including, but not limited to, kidney, heart, and lung, caused by rejection of the transplanted organ or tissue. In yet another embodiment, the hormone-treated fibroblasts (including fibroblast cells; fibroblast-like cells; and/or extracellular vesicles including exosomes, microvesicles, apototic bodies or any other fragments or biologic components of fibroblast cells) are administered to an individual to repair epithelial damage caused by burns, cuts, lacerations, and ulcerations, including, but not limited to, skin ulcerations and diabetic ulcerations.

### IV. Administration of Therapeutic Compositions

The therapy provided herein may comprise administration of any therapeutic agents described herein (e.g., fibroblasts, exosomes from fibroblasts, hormones, etc. (including fibroblast cells; fibroblast-like cells; and/or extracellular vesicles including exosomes, microvesicles, apototic bodies or any other fragments or biologic components of fibroblast cells)) alone or in combination. The therapy provided herein may comprise administration of a combination of therapeutic agents, such as a fibroblast therapy (including any fibroblast population disclosed herein) and a hormone therapy. The therapies may be administered in any suitable manner known in the art. For example, the fibroblast therapy and hormone therapy may be administered sequentially (at different times and in any order) or concurrently (at the same time). In some embodiments, the therapies are administered in a separate composition. In some embodiments, the therapies are in the same composition.

In some embodiments, the fibroblast therapy (including fibroblast cells; fibroblast-like cells; and/or extracellular vesicles including exosomes, microvesicles, apototic bodies or any other fragments or biologic components of fibroblast cells) and the hormone therapy are administered substantially simultaneously. In some embodiments, the fibroblast therapy and the hormone therapy are administered sequentially. In some embodiments, the fibroblast therapy and the hormone therapy and a third therapy are administered sequentially. In some embodiments, the fibroblast therapy is administered before administering the hormone therapy. In some embodiments, the fibroblast therapy is administered after administering the hormone therapy.

Any therapy, including the hormone-treated fibroblasts (including fibroblast cells; fibroblast-like cells; and/or extracellular vesicles including exosomes, microvesicles, apototic bodies or any other fragments or biologic components of fibroblast cells), may be administered systemically. In embodiments concerning the treatment of osteoarthritis or rheumatoid arthritis, hormone treated fibroblasts may be administered directly to an arthritic j oint. The hormone treated fibroblasts may be administered in an amount effective to treat an inflammatory response in an individual. The hormone treated fibroblasts may be administered in an amount of from about 1×10⁵ cells/kg to about 1×10⁷ cells/kg. In another embodiment, the hormone treated fibroblasts are administered in an amount of from about 1×10⁶ cells/kg to about 5×10⁶ cells/kg. The exact dosage of hormone treated fibroblasts to be administered is dependent upon a variety of factors, including the age, weight, and sex of the patient, the inflammatory response being treated, and the extent and severity thereof.

Therapies may be administered in any suitable manner known in the art. Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed. Any of the therapeutic agents (e.g., fibroblasts, hormones) of the disclosure may be administered by the same route of administration or by different routes of administration. In some embodiments, a therapy of the disclosure is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally.

The appropriate dosage may be determined based on the type of disease to be treated, severity and course of the disease, the clinical condition of the individual, the individual’s clinical history and response to the treatment, and the discretion of the attending physician. In some embodiments hormones, and/or hormone treated fibroblasts are administered together with a variety of therapeutic agents in order to enhance activity. Such therapeutic agents may be chosen from a group consisting of G-CSF, G-CSF′, flt3-Ligand, EGF′, FGF-1, FGF-2, IGF-1, VEGF, anti-TNF alpha antibodies, COX-2 inhibitors, anti-inflammatory agents, gold, cyclosporine, rapamycin, tacrolimus, and a combination thereof.

In some embodiments, the method further includes administering an additional therapeutic agent. In some embodiments, the additional therapeutic agent is administered prior to administration of cells. In some embodiments, the additional therapeutic agent is administered after administration of the cells. In some embodiments, the additional therapeutic agent is administered concurrently with administration of the cells. In some embodiments, the additional therapeutic agent is an immunosuppressive agent, a disease-modifying anti-rheumatic drug (DMARD), a biologic response modifier (a type of DMARD), a corticosteroid, or a nonsteroidal anti-inflammatory medication (NSAID), prednisone, prednisolone, methylprednisolone, methotrexate, hydroxychloroquine, sulfasalazine, leflunomide, cyclophosphamide, azathioprine, tofacitinib, adalimumab, abatacept, anakinra, kineret, certolizumab, etanercept, golimumab, infliximab, rituximab or tocilizumab, 6-mercaptopurine, 6-thioguanine, abatacept, adalimumab, alemtuzumab, an aminosalicylate, an antibiotic, an anti-histamine, Anti-TNFα, azathioprine, belimumab, beta interferon, a calcineurin inhibitor, certolizumab, a corticosteroid, cromolyn, cyclosporin A, cyclosporine, dimethyl fumarate, etanercept, fingolimod, fumaric acid esters, glatiramer acetate, golimumab, hydroxyurea, IFNy, IL-11, leflunomide, leukotriene receptor antagonist, long-acting beta2 agonist, mitoxantrone, mycophenolate mofetil, natalizumab, ocrelizumab, pimecrolimus, a probiotic, a retinoid, salicylic acid, short-acting beta2 agonist, sulfasalazine, tacrolimus, teriflunomide, theophylline, tocilizumab, ustekinumab, or vedolizumab, bevacuzimab, ranibizumab, or aflibercept), photodynamic therapy, photocoagulation, carbidopalevodopa, a dopamine agonist, an MAO-B inhibitor, a catechol-O-methyltransferase inhibitor, an anticholinergic, amantadine, deep brain stimulation, an anticoagulant, an anti-platelet agent, an angiotensin-converting enzyme inhibitor, an angiotensin II receptor blocker, an angiotensin receptor neprilysin inhibitor, a beta blocker, a combined alpha and beta blocker, a calcium channel blocker, a cholesterol lowering medication, a nicotinic acid, a cholesterol absorption inhibitor, a digitalis preparation, a diuretic, a vasodilator, a dual anti-platelet therapy, a cardiac procedure, an antiviral compound, a nucleoside-analog reverse transcriptase inhibitor (NRTI), a non-nucleoside reverse transcriptase inhibitor (NNRTI), a protease inhibitor, an antibacterial compound, an antifungal compound, an antiparasitic compound, insulin, a sulfonylurea, a biguanide, a meglitinide, a thiazolidinedione, a DPP-4 inhibitor, an SGLT2 inhibitor, an alpha-glucosidase inhibitor, a bile acid sequestrant, aspirin, a dietary regimen, a clotting factor, desmopressin, a clotpreserving medication, a fibrin sealant, physical therapy, a coenzyme, a bone marrow transplant, an organ transplant, hemodialysis, hemofiltration, exchange transfusion, peritoneal dialysis, medium-chain triacylglycerols, miglustat, enzyme supplementation therapy, a checkpoint inhibitor, a chemotherapeutic drug, a biologic drug, radiation therapy, cryotherapy, hyperthermia, surgical excision or tumor tissue, or an anti-cancer vaccine.

Furthermore, in some embodiments additional cells are utilized as therapeutics and administered systemically, locally, or peripherally together with fibroblasts that have been hormonally altered. The additional cells may be stem cells or progenitor cells (e.g., iPSC, embryonic stem cells, hematopoietic stem cells, mesenchymal stem cells, endothelial stem cells, epithelial stem cells, adipose stem or progenitor cells, germline stem cells, lung stem or progenitor cells, mammary stem cells, olfactory adult stem cells, hair follicle stem cells, multipotent stem cells, amniotic stem cells, cord blood stem cells, or neural stem or progenitor cells). In some embodiments, the stem cells are adult stem cells (e.g., somatic stem cells or tissue specific stem cells). In some embodiments, the stem or progenitor cell is capable of being differentiated (e.g., the stem cell is totipotent, pluripotent, or multipotent). In some embodiments, the cell is isolated from embryonic or neonatal tissue. In some embodiments, the additional cell is a fibroblast, monocytic precursor, B cell, exocrine cell, pancreatic progenitor, endocrine progenitor, hepatoblast, myoblast, preadipocyte, progenitor cell, hepatocyte, chondrocyte, smooth muscle cell, K562 human erythroid leukemia cell line, bone cell, synovial cell, tendon cell, ligament cell, meniscus cell, adipose cell, dendritic cells, or natural killer cell. In some embodiments, the cell is manipulated (e.g., converted or differentiated) into a muscle cell, erythroid-megakaryocytic cell, eosinophil, iPS cell, macrophage, T cell, islet beta-cell, neuron, cardiomyocyte, blood cell, endocrine progenitor, exocrine progenitor, ductal cell, acinar cell, alpha cell, beta cell, delta cell, PP cell, hepatocyte, cholangiocyte, or brown adipocyte. In some embodiments, the cell is a muscle cell (e.g., skeletal, smooth, or cardiac muscle cell), erythroid-megakaryocytic cell, eosinophil, iPS cell, macrophage, T cell, islet beta-cell, neuron, cardiomyocyte, blood cell (e.g., red blood cell, white blood cell, or platelet), endocrine progenitor, exocrine progenitor, ductal cell, acinar cell, alpha cell, beta cell, delta cell, PP cell, hepatocyte, cholangiocyte, or white or brown adipocyte. In some embodiments, the cell is a hormone-secreting cell (e.g., a cell that secretes insulin, oxytocin, endorphin, vasopressin, serotonin, somatostatin, gastrin, secretin, glucagon, thyroid hormone, bombesin, cholecystokinin, testosterone, estrogen, or progesterone, renin, ghrelin, amylin, or pancreatic polypeptide), an epidermal keratinocyte, an epithelial cell (e.g., an exocrine secretory epithelial cell, a thyroid epithelial cell, a keratinizing epithelial cell, a gall bladder epithelial cell, or a surface epithelial cell of the cornea, tongue, oral cavity, esophagus, anal canal, distal urethra, or vagina), a kidney cell, a germ cell, a skeletal joint synovium cell, a periosteum cell, a bone cell (e.g., osteoclast or osteoblast), a perichondrium cell (e.g., a chondroblast or chondrocyte), a cartilage cell (e.g., chondrocyte), a fibroblast, an endothelial cell, a pericardium cell, a meningeal cell, a keratinocyte precursor cell, a keratinocyte stem cell, a pericyte, a glial cell, an ependymal cell, a cell isolated from an amniotic or placental membrane, or a serosal cell (e.g., a serosal cell lining body cavities). In some embodiments, the cell is a somatic cell. In some embodiments, the cells are derived from skin or other organs, e.g., heart, brain or spinal cord, liver, lung, kidney, pancreas, bladder, bone marrow, spleen, intestine, or stomach. The cells can be from humans or other mammals (e.g., rodent, non-human primate, bovine, or porcine cells). It is contemplated herein that hormone-treated fibroblasts may be of use in cell-based therapies wherein it may be desirable to evade allorejection at a localized transplant site.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

In some embodiments, the fibroblast population (including fibroblast cells; fibroblast-like cells; and/or extracellular vesicles including exosomes, microvesicles, apototic bodies or any other fragments or biologic components of fibroblast cells) and/or the hormone is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg.

In some embodiments, a single dose of the fibroblast therapy and/or hormone therapy is administered. In some embodiments, multiple doses of the fibroblast therapy and/or hormone therapy are administered. In some embodiments, the fibroblast therapy and/or hormone therapy is administered at a dose of between 1 mg/kg and 100 mg/kg. In some embodiments, the fibroblast therapy and/or hormone therapy is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 µg/kg, mg/kg, µg/day, or mg/day or any range derivable therein. Furthermore, such doses can be administered at multiple times during a day, and/or on multiple days, weeks, or months.

In certain embodiments, the effective dose of the hormone therapy is one which can provide a blood level of about 1 µM to 150 µM. In another embodiment, the effective dose provides a blood level of about 4 µM to 100 µM.; or about 1 µM to 100 µM; or about 1 µM to 50 µM; or about 1 µM to 40 µM; or about 1 µM to 30 µM; or about 1 µM to 20 µM; or about 1 µM to 10 µM; or about 10 µM to 150 µM; or about 10 µM to 100 µM; or about 10 µM to 50 µM; or about 25 µM to 150 µM; or about 25 µM to 100 µM; or about 25 µM to 50 µM; or about 50 µM to 150 µM; or about 50 µM to 100 µM (or any range derivable therein). In other embodiments, the dose can provide the following blood level of the hormone being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 µM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of µg/kg or mg/kg of body weight can be converted and expressed in comparable concentration units of µg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The active compounds can be formulated for parenteral administration, e.g., formulated for injection via the intravenous, intramuscular, subcutaneous, or intraperitoneal routes. Typically, such compositions may be prepared as either liquid solutions or suspensions; solid forms suitable for use to prepare solutions or suspensions upon the addition of a liquid prior to injection may also be prepared; the preparations may also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, isotonic agents are included, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the methods of preparation include vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### V. Cellular Therapies

### A. Cell Culture

In some embodiments, a nutrient medium comprises any of the following in an appropriate combination: isotonic saline, buffer, amino acids, antibiotics, serum or serum replacement, and exogenously added factors. Amniotic fluid fibroblast cells may be grown in an undifferentiated state for as long as desired (and optionally stored as described above), and can then be cultured under certain conditions to allow progression to a differentiated state. While it is known that once sufficient cellular mass is achieved, cells can be differentiated into endodermal, mesodermal and ectodermal derived tissues in vitro and in vivo. This planned, specialized differentiation from undifferentiated cells towards a specific cell type or tissue type is termed “directed differentiation.” Exemplary cell types that may be prepared from regenerative cells using directed differentiation toward anti-inflammatory phenotype include but are not limited to derivation of cells possessing CD105 associated with cells selected from a group comprising of: fat cells, cardiac muscle cells, epithelial cells, liver cells, brain cells, blood cells, neurons, glial cells, pancreatic cells, and the like.

In some embodiments, cells are cultured for at least between about 1 day and about 40 days, for at least between about 15 days and about 35 days, for at least between about 15 days and 21 days, such as for at least about 15, 16, 17, 18, 19 or 21 days. In some embodiments, the cells of the disclosure are cultured for no longer than 60 days, or no longer than 50 days, or no longer than 45 days. The cells may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days. The cells may be cultured in the presence of a liquid culture medium. Typically, the medium may comprise a basal medium formulation as known in the art. Many basal media formulations can be used to culture cells herein, including but not limited to Eagle’s Minimum Essential Medium (MEM), Dulbecco’s Modified Eagle’s Medium (DMEM), alpha modified Minimum Essential Medium (alpha-MEM), Basal Medium Essential (BME), Iscove’s Modified Dulbecco’s Medium (IMDM), BGJb medium, F-12 Nutrient Mixture (Ham), Liebovitz L-15, DMEM/F-12, Essential Modified Eagle’s Medium (EMEM), RPMI-1640, and modifications and/or combinations thereof. Compositions of the above basal media are generally known in the art, and it is within the skill of one in the art to modify or modulate concentrations of media and/or media supplements as necessary for the cells cultured. In some embodiments, a culture medium formulation may be explants medium (CEM) which is composed of IMDM supplemented with 10% fetal bovine serum (FBS), 100 U/ml penicillin G, 100 µg/ml streptomycin and 2 mmol/L L-glutamine. Other embodiments may employ further basal media formulations, such as chosen from the ones above.

Any medium capable of supporting cells in vitro may be used to culture the cells. Media formulations that can support the growth of cells include, but are not limited to, Dulbecco’s Modified Eagle’s Medium (DMEM), alpha modified Minimal Essential Medium (αMEM), and Roswell Park Memorial Institute Media 1640 (RPMI Media 1640) and the like. Typically, up to 20% fetal bovine serum (FBS) or 1-20% horse serum is added to the above medium in order to support the growth of cells. A defined medium, however, also can be used if the growth factors, cytokines, and hormones necessary for culturing cells are provided at appropriate concentrations in the medium. Media useful in the methods of the disclosure may comprise one or more compounds of interest, including, but not limited to, antibiotics, mitogenic compounds, or differentiation compounds useful for the culturing of cells. The cells may be grown at temperatures between 27° C. to 40° C., such as 31° C. to 37° C., and may be in a humidified incubator. The carbon dioxide content may be maintained between 2% to 10% and the oxygen content may be maintained between 1% and 22%. The disclosure, however, should in no way be construed to be limited to any one method of isolating and culturing cells. Rather, any method of isolating and culturing cells should be construed to be included in the present disclosure.

For use in the cell culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks’ Balanced Salt Solution (HBSS), Earle’s Salt Solution. Further antioxidant supplements may be added, e.g., β-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In particular embodiments, cells are cultured in a cell culture system comprising a cell culture medium, including in a culture vessel, in particular a cell culture medium supplemented with a substance suitable and determined for protecting the cells from in vitro aging and/or inducing in an unspecific or specific reprogramming.

### B. Cell Generation

Certain methods of the disclosure concern culturing the cells obtained from human tissue samples. In particular embodiments of the present disclosure, cells are plated onto a substrate that allows for adherence of cells thereto. This may be carried out, for example, by plating the cells in a culture plate that displays one or more substrate surfaces compatible with cell adhesion. When the one or more substrate surfaces contact the suspension of cells (e.g., suspension in a medium) introduced into the culture system, cell adhesion between the cells and the substrate surfaces may ensue. Accordingly, in certain embodiments cells are introduced into a culture system that features at least one substrate surface that is generally compatible with adherence of cells thereto, such that the plated cells can contact the said substrate surface, such embodiments encompass plating onto a substrate, which allows adherence of cells thereto.

The fibroblasts utilized in the disclosure are generated, in one embodiment, by outgrowth from a biopsy of the recipient’s own skin (in the case of autologous preparations), or skin of healthy donors (for allogeneic preparations). In some embodiments, fibroblasts are used from young donors. In another embodiment, fibroblasts are transfected with genes to allow for enhanced growth and overcoming of the Hayflick limit. Subsequent to derivation of cells expansion in culture using standard cell culture techniques. Skin tissue (dermis and epidermis layers) may be biopsied from a subject’s post-auricular area. In one embodiment, the starting material is composed of three 3-mm punch skin biopsies collected using standard aseptic practices. The biopsies are collected by the treating physician, placed into a vial containing sterile phosphate buffered saline (PBS). The biopsies are shipped in a 2-8° C. refrigerated shipper back to the manufacturing facility. In one embodiment, after arrival at the manufacturing facility, the biopsy is inspected and, upon acceptance, transferred directly to the manufacturing area. Upon initiation of the process, the biopsy tissue is then washed prior to enzymatic digestion. After washing, a Liberase Digestive Enzyme Solution is added without mincing, and the biopsy tissue is incubated at 37.0 ± 2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other commercially available collagenases may be used, such as Serva Collagenase NB6 (Helidelburg, Germany). After digestion, Initiation Growth Media (IMDM, GA, 10% Fetal Bovine Serum (FBS)) is added to neutralize the enzyme, cells are pelleted by centrifugation and resuspended in 5.0 mL Initiation Growth Media. Alternatively, centrifugation is not performed, with full inactivation of the enzyme occurring by the addition of Initiation Growth Media only. Initiation Growth Media is added prior to seeding of the cell suspension into a T-175 cell culture flask for initiation of cell growth and expansion. A T-75, T-150, T-185 or T-225 flask can be used in place of the T-75 flask. Cells are incubated at 37 ± 2.0° C. with 5.0 ± 1.0% CO₂ and fed with fresh Complete Growth Media every three to five days. All feeds in the process are performed by removing half of the Complete Growth Media and replacing the same volume with fresh media. Alternatively, full feeds can be performed. Cells should not remain in the T-175 flask greater than 30 days prior to passaging. Confluence is monitored throughout the process to ensure adequate seeding densities during culture splitting. When cell confluence is greater than or equal to 40% in the T-175 flask, they are passaged by removing the spent media, washing the cells, and treating with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask. Morphology is evaluated at each passage and prior to harvest to monitor the culture purity throughout the culture purity throughout the process. Morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures is also evaluated. Keratinocytes appear round and irregularly shaped and, at higher confluence, they appear organized in a cobblestone formation. At lower confluence, keratinocytes are observable in small colonies. Cells are incubated at 37 ± 2.0° C. with 5.0 ± 1.0% CO2 and passaged every three to five days in the T-500 flask and every five to seven days in the ten layer cell stack (10CS). Cells should not remain in the T-500 flask for more than 10 days prior to passaging. Quality Control (QC) release testing for safety of the Bulk Drug Substance includes sterility and endotoxin testing. When cell confluence in the T-500 flask is >95%, cells are passaged to a 10 CS culture vessel. Alternately, two Five Layer Cell Stacks (5 CS) or a 10 Layer Cell Factory (10 CF) can be used in place of the 10 CS. Passage to the 10 CS is performed by removing the spent media, washing the cells, and treating with Trypsin-EDTA to release adherent cells in the flask into the solution. Cells are then transferred to the 10 CS. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers. Cells are then incubated at 37 ± 2.0° C. with 5.0 ± 1.0% CO₂ and fed with fresh Complete Growth Media every five to seven days. Cells should not remain in the 10CS for more than 20 days prior to passaging. In one embodiment, the passaged dermal fibroblasts are rendered substantially free of immunogenic proteins present in the culture medium by incubating the expanded fibroblasts for a period of time in protein free medium, Primary Harvest When cell confluence in the 10 CS is 95% or more, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process QC testing performed to determine total viable cell count and cell viability.

Cells of the present disclosure may be identified and characterized by their expression of specific marker proteins, such as cell-surface markers. Detection and isolation of these cells can be achieved, for example, through flow cytometry, ELISA, and/or magnetic beads. Reversetranscription polymerase chain reaction (RT-PCR) may be used to quantify cell-specific genes and/or to monitor changes in gene expression in response to differentiation. In certain embodiments, the marker proteins used to identify and characterize the cells are selected from the list consisting of c-Kit, Nanog, Sox2, Hey1, SMA, Vimentin, Cyclin D2, Snail, E-cadherin, Nkx2.5, GATA4, CD105, CD90, CD29, CD73, Wt1, CD34, CD45, and a combination thereof.

### C. Pharmaceutical Compositions

In certain aspects, the compositions or agents for use in the methods, such as one or more hormones or hormone-like compositions and/or a population of fibroblasts, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

In some embodiments, the fibroblast dosage formulation is an autologous cell therapy product composed of a suspension of autologous fibroblasts, grown from a biopsy of each individual’s own skin using standard tissue culture procedures.

Suitable carriers for parenteral delivery via injectable, infusion or irrigation and topical delivery include distilled water, physiological phosphate-buffered saline, normal or lactated Ringer’s solutions, dextrose solution, Hank’s solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of nonlimiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer’s dextrose, etc. Intravenous vehicles include fluid and nutrient replenishers. Preservatives include antimicrobial agents, antgifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 mL and 0.5 mL, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference
1. Zhang, H., et al., Dihydrotestosterone modulates endothelial progenitor cell function via RhoA/ROCKpathway. Am J Transl Res, 2016. 8(10): p. 4300-4309.
2. Ray, R., et al., Sex steroids and stem cell function. Mol Med, 2008. 14(7-8): p. 493-501.
3. Fadini, G.P., et al., Effects of androgens on endothelial progenitor cells in vitro and in vivo. Clin Sci (Lond), 2009. 117(10): p. 355-64.
4. La Vignera, S., et al., Original immunophenotype of blood endothelial progenitor cells and microparticles in patients with isolated arterial erectile dysfunction and late onset hypogonadism: effects of androgen replacement therapy. Aging Male, 2011. 14(3): p. 183-9.
5. Foresta, C., et al., Androgens stimulate endothelial progenitor cells through an androgen receptor-mediated pathway. Clin Endocrinol (Oxf), 2008. 68(2): p. 284-9.
6. Foresta, C., et al., Reduced number of circulating endothelial progenitor cells in hypogonadal men. J Clin Endocrinol Metab, 2006. 91(11): p. 4599-602.
7. Foresta, C., et al., Effect of vardenafil on endothelial progenitor cells in hypogonadotrophic hypogonadal patients: role of testosterone treatment. Clin Endocrinol (Oxf), 2009. 71(3): p. 412-6.
8. Rousseau, A., et al., Impact of age and gender interaction on circulating endothelial progenitor cells in healthy subjects. Fertil Steril, 2010. 93(3): p. 843-6.
9. Florvaag, A., et al., Testosterone deficiency in male heart failure patients and its effect on endothelial progenitor cells. Aging Male, 2012. 15(3): p. 180-6.
10. Barud, W., et al., Association of stromal-derived factor-1 alpha and endogenous sex hormones in men aged over 50 years with stable coronary artery disease. Adv Med Sci, 2012. 57(2): p. 322-7.
11. Omar, Y.A., et al., Testosterone level and endothelial dysfunction in patients with vasculogenic erectile dysfunction. Andrology, 2017. 5(3): p. 527-534.
12. Topel, M.L., et al., Sex Differences in Circulating Progenitor Cells. J Am Heart Assoc, 2017. 6(10).
13. Hotta, Y., T. Kataoka, and K. Kimura, Testosterone Deficiency and Endothelial Dysfunction: Nitric Oxide, Asymmetric Dimethylarginine, and Endothelial Progenitor Cells. Sex Med Rev, 2019. 7(4): p. 661-668.
14. Liao, C.H., et al., Testosterone replacement therapy can increase circulating endothelial progenitor cell number in men with late onset hypogonadism. Andrology, 2013. 1(4): p. 563-9.
15. Hwang, I., et al., Testosterone modulates endothelial progenitor cells in rat corpus cavernosum. BJU Int, 2016. 117(6): p. 976-81.
16. Corotchi, M.C., M.A. Popa, and M. Simionescu, Testosterone stimulates proliferation and preserves sternness of human adult mesenchymal stem cells and endothelial progenitor cells. Rom J Morphol Embryol, 2016. 57(1): p. 75-80.
17. Skurikhin, E.G., et al., Response of Stem and Progenitor Cells to Testicular Ischemia. Bull Exp Biol Med, 2016. 161(4): p. 523-8.
18. Motamer, M., et al., Evaluation the effect of testosterone on the number of endothelial progenitor cells and amount of SDF-lalpha, PDGF, bFGF, and NO. Int J Prev Med, 2019. 10: p. 214.
19. Lam, Y.T., et al., Androgens Stimulate EPC-Mediated Neovascularization and Are Associated with Increased Coronary Collateralization. Endocrinology, 2020. 161(5).
20. Byron, J.W., Comparison of the action of 3 H-thymidine and hydroxyurea on testosterone-treated hemopoietic stem cells. Blood, 1972. 40(2): p. 198-203.
21. Jepson, J.H., et al., Effect of androgenic steroids on erythropoiesis. Biomedicine, 1973. 19(3): p. 83-6.
22. Jepson, J.H., et al., Current concepts of the action of androgenic steroids on erythropoiesis. J Pediatr, 1973. 83(4): p. 703-8.
23. Airoldi, G., et al., [Experimental studies on the effects of testosterone on bone marrow cellularity in albino rats, with special reference to the stem cell problem]. Riv Emoter Immunoematol, 1974. 21(1-2): p. 35-53.
24. Moriyama, Y. and J.W. Fisher, Effects of testosterone and erythropoietin on erythroid colony formation in rabbit bone marrow cultures. Life Sci, 1974. 15(6): p. 1181-8.
25. Malgor, L.A., L. Barrios, and C.C. Blanc, Effects of testosterone on bone marrow erythroid cells of normal and nephrectomized rats. Acta Physiol Lat Am, 1975. 25(3): p. 179-87.
26. Moriyama, Y. and J.W. Fisher, Increase in erythroid colony formation in rabbits following the administration of testosterone. Proc Soc Exp Biol Med, 1975. 149(1): p. 178-80.
27. Beran, M., G. Spitzer, and D.S. Verma, Testosterone and synthetic and androgens improve the in vitro survival of human marrow progenitor cells in serum-free suspension cultures. J Lab Clin Med, 1982. 99(2): p. 247-53.
28. Bogliolo, G., et al., Studies on hemopoietic stem cells in mice treated with testosterone propionate and anticancer drugs. Tumori, 1982. 68(4): p. 277-82.
29. Modder, B. and G. Futterer, Protective effect of testosterone or 5 beta-dihydrotestosterone pretreatment on CFU-E numbers in busulfan-treated rabbits. Cancer Chemother Pharmacol, 1985. 15(3): p. 236-9.
30. Claustres, M. and C. Sultan, Androgen and erythropoiesis: evidence for an androgen receptor in erythroblasts from human bone marrow cultures. Horm Res, 1988. 29(1): p. 17-22.
31. Nissen, C., et al., Testosterone reduces complement sensitivity of precursor cells in aplastic anaemia patients treated with antilymphocyte globulin. Br J Haematol, 1988. 69(3): p. 405-11.
32. Worthington-White, D.A. and S. Gross, Estrogen blocks the cimetidine-induced suppression of CFU-GM. Exp Hematol, 1993. 21(1): p. 16-20.
33. Mirand, E.A. and A.S. Gordon, Mechanism of estrogen action in erythropoiesis. Endocrinology, 1966. 78(2): p. 325-32. 34. Kubanek, B., Erythropoietin: the haematologist’s hormone. Horm Metab Res, 1969. 1(4): p. 151-6.
35. Cruickshank, J.M., The relationship of urinary total oestrogens with haemoglobin concentration. J Obstet Gynaecol Br Commonw, 1970. 77(7): p. 640-3.
36. Peschle, C., et al., The role of estrogen in the regulation of erythropoietin production. Endocrinology, 1973. 92(2): p. 358-62.
37. Fried, W., et al., Effects of estrogens on hematopoietic stem cells and on hematopoiesis of mice. J Lab Clin Med, 1974. 83(5): p. 807-15.
38. Adler, S.S. and F.E. Trobough, Jr., Effects of estrogen on erythropoiesis and granuloid progenitor cell (CFU-C) proliferation in mice. J Lab Clin Med, 1978. 91(6): p. 960-8.
39. Crandall, T.L., R.A. Joyce, and D.R. Boggs, Estrogens and hematopoiesis: characterization and studies on the mechanism of neutropenia. J Lab Clin Med, 1980. 95(6): p. 857-67.
40. Hayama, T., et al., Effects of estrogen on hepatic hemopoiesis in the adult mouse. Exp Hematol, 1983. 11(7): p. 611-7.
41. Manolagas, S.C. and R.L. Jilka, Cytokines, hematopoiesis, osteoclastogenesis, and estrogens. Calcif Tissue Int, 1992. 50(3): p. 199-202.
42. Kincade, P.W., K.L. Medina, and G. Smithson, Sex hormones as negative regulators of lymphopoiesis. Immunol Rev, 1994. 137: p. 119-34.
43. Issa, J.P., et al., The estrogen receptor CpG island is methylated in most hematopoietic neoplasms. Cancer Res, 1996. 56(5): p. 973-77.
44. Aronica, S.M., et al., Altered bone marrow cell sensitivity in the lupus-prone NZB/W mouse: regulation of CFU-GM colony formation by estrogen, tamoxifen and thrombopoietin. Lupus, 2000. 9(4): p. 271-7.
45. Benayahu, D., I. Shur, and S. Ben-Eliyahu, Hormonal changes affect the bone and bone marrow cells in a rat model. J Cell Biochem, 2000. 79(3): p. 407-15.
46. Perry, M.J., et al., Effects of high-dose estrogen on murine hematopoietic bone marrow precede those on osteogenesis. Am J Physiol Endocrinol Metab, 2000. 279(5): p. E1159-65.
47. Verthelyi, D., Sex hormones as immunomodulators in health and disease. Int Immunopharmacol, 2001. 1(6): p. 983-93.
48. Medina, K.L., et al., Identification of very early lymphoid precursors in bone marrow and their regulation by estrogen. Nat Immunol, 2001. 2(8): p. 718-24.
49. Erben, R.G., J. Eberle, and M. Stangassinger, B lymphopoiesis is upregulated after orchiectomy and is correlated with estradiol but not testosterone serum levels in aged male rats. Horm Metab Res, 2001. 33(8): p. 491-8.
50. Cammenga, J., et al., Induction of C/EBPalpha activity alters gene expression and differentiation of human CD34+ cells. Blood, 2003. 101(6): p. 2206-14.
51. Erlandsson, M.C., et al., Oestrogen receptor specificity in oestradiol-mediated effects on B lymphopoiesis and immunoglobulin production in male mice. Immunology, 2003. 108(3): p. 346-51.
52. Shim, G.J., et al., Disruption of the estrogen receptor beta gene in mice causes myeloproliferative disease resembling chronic myeloid leukemia with lymphoid blast crisis. Proc Natl Acad Sci U S A, 2003. 100(11): p. 6694-9.
53. Islander, U., et al., Influence of oestrogen receptor alpha and beta on the immune system in aged female mice. Immunology, 2003. 110(1): p. 149-57.
54. Pittenger, G.L., A.I. Vinik, and L. Rosenberg, The partial isolation and characterization of ilotropin, a novel islet-specific growth factor. Adv Exp Med Biol, 1992. 321: p. 123-30; discussion 131-2.
55. Nielsen, J.H., et al., The role of growth hormone and prolactin in beta cell growth and regeneration. Adv Exp Med Biol, 1992. 321: p. 9-17; discussion 19-20.
56. Movassat, J., C. Saulnier, and B. Portha, Insulin administration enhances growth of the beta-cell mass in streptozotocin-treated newborn rats. Diabetes, 1997. 46(9): p. 1445-52.
57. Holstad, M. and S. Sandler, Prolactin protects against diabetes induced by multiple low doses of streptozotocin in mice. J Endocrinol, 1999. 163(2): p. 229-34.
58. Xu, G., et al., Exendin-4 stimulates both beta-cell replication and neogenesis, resulting in increased beta-cell mass and improved glucose tolerance in diabetic rats. Diabetes, 1999. 48(12): p. 2270-6.
59. Cui, H., et al., Safety, Pharmacokinetics and Pharmacodynamics of Multiple Escalating Doses of PEGylated Exenatide (PB-119) in Healthy Volunteers. Eur J Drug Metab Pharmacokinet, 2021. 46(2): p. 265-275.
60. Song, X., et al., Combined Treatment with Bone Marrow -Derived Mesenchymal Stem Cells and Exendin-4 Promotes Islet Regeneration in Streptozotocin-Induced Diabetic Rats. Stem Cells Dev, 2021. 30(9): p. 502-514.
61. Tourrel, C., et al., Glucagon-like peptide-1 and exendin-4 stimulate beta-cell neogenesis in streptozotocin-treated newborn rats resulting in persistently improved glucose homeostasis at adult age. Diabetes, 2001. 50(7): p. 1562-70.
62. Bakbak, E., et al., Lessons from bariatric surgery: Can increased GLP-1 enhance vascular repair during cardiometabolic-based chronic disease? Rev Endocr Metab Disord, 2021.
63. Abdulmalik, S., et al., The glucagon-like peptide 1 receptor agonist Exendin-4 induces tenogenesis in human mesenchymal stem cells. Differentiation, 2021. 120: p. 1-9.
64. Mehta, K., et al., Deciphering the Neuroprotective Role of Glucagon-like Peptide-1 Agonists in Diabetic Neuropathy: Current Perspective and Future Directions. Curr Protein Pept Sci, 2021. 22(1): p. 4-18.
65. Villalba, A., et al., Repurposed Analog of GLP-1 Ameliorates Hyperglycemia in Type 1 Diabetic Mice Through Pancreatic Cell Reprogramming. Front Endocrinol (Lausanne), 2020. 11: p. 258.
66. Wei, R., et al., Dapagliflozin promotes beta cell regeneration by inducing pancreatic endocrine cell phenotype conversion in type 2 diabetic mice. Metabolism, 2020. 111: p. 154324.
67. Li, L., et al., Promotion of beta-cell regeneration by betacellulin in ninety percent-pancreatectomized rats. Endocrinology, 2001. 142(12): p. 5379-85.
68. Li, L., et al., Betacellulin improves glucose metabolism by promoting conversion of intraisletprecursor cells to beta-cells in streptozotocin-treated mice. Am J Physiol Endocrinol Metab, 2003. 285(3): p. E577-83.
69. Lee, Y.S., G.J. Song, and H.S. Jun, Betacellulin-Induced alpha-Cell Proliferation Is Mediated by ErbB3 and ErbB4, and May Contribute to beta-Cell Regeneration. Front Cell Dev Biol, 2020. 8: p. 605110.
70. Li, L., et al., Activin A and betacellulin: effect on regeneration of pancreatic beta-cells in neonatal streptozotocin-treated rats. Diabetes, 2004. 53(3): p. 608-15.
71. Rooman, I., J. Lardon, and L. Bouwens, Gastrin stimulates beta-cell neogenesis and increases islet mass from transdifferentiated but not from normal exocrine pancreas tissue. Diabetes, 2002. 51(3): p. 686-90.
72. Rooman, I. and L. Bouwens, Combined gastrin and epidermal growth factor treatment induces islet regeneration and restores normoglycaemia in C57B16/J mice treated with alloxan. Diabetologia, 2004. 47(2): p. 259-65.
73. George, M., et al., Beta cell expression of IGF-I leads to recovery from type 1 diabetes. J Clin Invest, 2002. 109(9): p. 1153-63.
74. Agudo, J., et al., IGF-I mediates regeneration of endocrine pancreas by increasing beta cell replication through cell cycle protein modulation in mice. Diabetologia, 2008. 51(10): p. 1862-72.
75. Sharma, A., et al., The homeodomain protein IDX-1 increases after an early burst of proliferation during pancreatic regeneration. Diabetes, 1999. 48(3): p. 507-13.
76. Watanabe, T., et al., Pancreatic beta-cell replication and amelioration of surgical diabetes by Reg protein. Proc Natl Acad Sci U S A, 1994. 91(9): p. 3589-92.
77. Okamoto, H., The Reg gene family and Reg proteins: with special attention to the regeneration of pancreatic beta-cells. J Hepatobiliary Pancreat Surg, 1999. 6(3): p. 254-62.
78. Kobayashi, S., et al., Identification of a receptor for reg (regenerating gene) protein, a pancreatic beta-cell regeneration factor. J Biol Chem, 2000. 275(15): p. 10723-6.
79. Kodama, M., et al., Embryonic stem cell transplantation correlates with endogenous neurogenin 3 expression and pancreas regeneration in streptozotocin-injured mice. J Histochem Cytochem, 2009. 57(12): p. 1149-58.
80. Magenheim, J., et al., Ngn3(+) endocrine progenitor cells control the fate and morphogenesis of pancreatic ductal epithelium. Dev Biol, 2011. 359(1): p. 26-36.
81. Jiang, F.X. and G. Morahan, Pancreatic stem cells: from possible to probable. Stem Cell Rev Rep, 2012. 8(3): p. 647-57.
82. Gribben, C., et al., Ductal Ngn3-expressingprogenitors contribute to adult beta cell neogenesis in the pancreas. Cell Stem Cell, 2021.
83. Zbinden, A., et al., Nidogen-1 Mitigates Ischemia and Promotes Tissue Survival and Regeneration. Adv Sci (Weinh), 2021. 8(4): p. 2002500.
84. Zhang, H., et al., Multiple, temporal-specific roles for HNF6 in pancreatic endocrine and ductal differentiation. Mech Dev, 2009. 126(11-12): p. 958-73.
85. Carpino, G., et al., Progenitor cell niches in the human pancreatic duct system and associated pancreatic duct glands: an anatomical and immunophenotyping study. J Anat, 2016. 228(3): p. 474-86.
86. Rezanejad, H., et al., Heterogeneity of SOX9 and HNF1beta in Pancreatic Ducts Is Dynamic. Stem Cell Reports, 2018. 10(3): p. 725-738.
87. Takahashi, I., et al., Important role of heparan sulfate in postnatal islet growth and insulin secretion. Biochem Biophys Res Commun, 2009. 383(1): p. 113-8.
88. Sachs, S., et al., Targeted pharmacological therapy restores beta-cell function for diabetes remission. Nat Metab, 2020. 2(2): p. 192-209.
89. Pittenger, G.L., D. Taylor-Fishwick, and A.I. Vinik, The role of islet neogeneis-associated protein (INGAP) in pancreatic islet neogenesis. Curr Protein Pept Sci, 2009. 10(1): p. 37-45.
90. Kapur, R., et al., Short-term effects of INGAP and Reg family peptides on the appearance of small beta-cells clusters in non-diabetic mice. Islets, 2012. 4(1): p. 40-8.
91. Kerem, M., et al., Exogenous ghrelin enhances endocrine and exocrine regeneration in pancreatectomized rats. J Gastrointest Surg, 2009. 13(4): p. 775-83.
92. Kayali, A.G., et al., The SDF-1alpha/CXCR4 axis is required for proliferation and maturation of human fetal pancreatic endocrine progenitor cells. PLoS One, 2012. 7(6): p. e38721.
93. Koya, V., et al., Reversal of streptozotocin-induced diabetes in mice by cellular transduction with recombinant pancreatic transcription factor pancreatic duodenal homeobox-1: a novel protein transduction domain-based therapy. Diabetes, 2008. 57(3): p. 757-69.
94. Luo, L., J.Z. Luo, and I.M. Jackson, Thyrotropin-releasing hormone (TRH) reverses hyperglycemia in rat. Biochem Biophys Res Commun, 2008. 374(1): p. 69-73.
95. Gandellini, P., et al., Complexity in the tumour microenvironment: Cancer associated fibroblast gene expression patterns identify both common and unique features of tumour-stroma crosstalk across cancer types. Semin Cancer Biol, 2015. 35: p. 96-106.
96. Cheung, P.K., et al., Protein profiling of microdissected prostate tissue links growth differentiation factor 15 to prostate carcinogenesis. Cancer Res, 2004. 64(17): p. 5929-33.
97. Suesskind, D., et al., GDF-15: a novel serum marker for metastases in uveal melanoma patients. Graefes Arch Clin Exp Ophthalmol, 2012. 250(6): p. 887-95.
98. Breit, S.N., et al., Macrophage inhibitory cytokine-1 (MIC-⅟GDF15) and mortality in end-stage renal disease. Nephrol Dial Transplant, 2012. 27(1): p. 70-5.
99. Brown, D.A., et al., Serum macrophage inhibitory cytokine-1 (MIC-⅟GDF15): a potential screening tool for the prevention of colon cancer? Cancer Epidemiol Biomarkers Prev, 2012. 21(2): p. 337-46.
100. Corre, J., et al., Bioactivity and prognostic significance of growth differentiation factor GDF15 secreted by bone marrow mesenchymal stem cells in multiple myeloma. Cancer Res, 2012. 72(6): p. 1395-406.
101. Schiegnitz, E., et al., GDF 15 as an anti-apoptotic, diagnostic and prognostic marker in oral squamous cell carcinoma. Oral Oncol, 2012. 48(7): p. 608-14.
102. Mimeault, M., S.L. Johansson, and S.K. Batra, Pathobiological implications of the expression of EGFR, pAkt, NF-kappaB and MIC-1 in prostate cancer stem cells and their progenies. PLoS One, 2012. 7(2): p. e31919.
103. Jin, Y.J., et al., Macrophage inhibitory cytokine-1 stimulates proliferation of human umbilical vein endothelial cells by up-regulating cyclins D1 and E through the PI3K/Akt-, ERK-, and JNK-dependent AP-1 and E2F activation signaling pathways. Cell Signal, 2012. 24(8): p. 1485-95.
104. Wang, X., S.J. Baek, and T.E. Eling, The diverse roles of nonsteroidal anti-inflammatory drug activated gene (NAG-⅟GDF15) in cancer. Biochem Pharmacol, 2013. 85(5): p. 597-606.
105. Khaled, Y.S., E. Elkord, and B.J. Ammori, Macrophage inhibitory cytokine-1: a review of its pleiotropic actions in cancer. Cancer Biomark, 2012. 11(5): p. 183-90.
106. Shimizu, S., et al., Proteasome inhibitor MG132 induces NAG-⅟GDF15 expression through the p38 MAPKpathway in glioblastoma cells. Biochem Biophys Res Commun, 2013. 430(4): p. 1277-82.
107. Kaur, S., et al., Potentials of plasma NGAL and MIC-1 as biomarker(s) in the diagnosis of lethal pancreatic cancer. PLoS One, 2013. 8(2): p. e55171.
108. Yang, C.Z., et al., Elevated level of serum growth differentiation factor 15 is associated with oral leukoplakia and oral squamous cell carcinoma. J Oral Pathol Med, 2014. 43(1): p. 28-34.
109. Unsicker, K., B. Spittau, and K. Krieglstein, The multiple facets of the TGF-betafamily cytokine growth/differentiationfactor-151macrophage inhibitory cytokine-1. Cytokine Growth Factor Rev, 2013. 24(4): p. 373-84.
110. Arslan, D., et al., Growth-differentiation factor-15 and tissue doppler imaging in detection of asymptomatic anthracycline cardiomyopathy in childhood cancer survivors. Clin Biochem, 2013. 46(13-14): p. 1239-43.
111. Aw Yong, K.M., et al., Morphological effects on expression of growth differentiation factor 15 (GDFIS), a marker of metastasis. J Cell Physiol, 2014. 229(3): p. 362-73.
112. Monteiro-Reis, S., et al., Accurate detection of upper tract urothelial carcinoma in tissue and urine by means of quantitative GDF15, TMEFF2 and VIM promoter methylation. Eur J Cancer, 2014. 50(1): p. 226-33.
113. Griner, S.E., et al., Mechanisms of Adipocytokine-Mediated Trastuzumab Resistance in HER2-Positive Breast Cancer Cell Lines. Curr Pharmacogenomics Person Med, 2013. 11(1): p. 31-41.
114. Corre, J., B. Hebraud, and P. Bourin, Concise review: growth differentiation factor 15 in pathology: a clinical role? Stem Cells Transl Med, 2013. 2(12): p. 946-52.
115. Corre, J., Growth differentiation factor 15 in multiple myeloma: a microenvironment factor predictive of response to treatment? Acta Haematol, 2014. 131(3): p. 170-2.
116. Tarkun, P., et al., Serum growth differentiation factor 15 levels in newly diagnosed multiple myeloma patients. Acta Haematol, 2014. 131(3): p. 173-8.
117. Wang, Z., et al., GDF15 induces immunosuppression via CD48 on regulatory T cells in hepatocellular carcinoma. J Immunother Cancer, 2021. 9(9).
118. Lodi, R.S., et al., Roles and Regulation of Growth differentiation factor-15 in the Immune and tumor microenvironment. Hum Immunol, 2021.
119. Mielcarska, S., et al., GDF-15 Level Correlates with CMKLR1 and VEGF-A in Tumor-free Margin in Colorectal Cancer. Curr Med Sci, 2021. 41(3): p. 522-528.
120. Song, J., et al., A panel of selected serum protein biomarkers for the detection of aggressive prostate cancer. Theranostics, 2021. 11(13): p. 6214-6224.
121. Fang, Z., et al., Risk prediction models for colorectal cancer: Evaluating the discrimination due to added biomarkers. Int J Cancer, 2021. 149(5): p. 1021-1030.
122. Kaneto, H., et al., Multifaceted Mechanisms of Action of Metformin Which Have Been Unraveled One after Another in the Long History. Int J Mol Sci, 2021. 22(5).
123. Rybicki, B.A., et al., Growth and differentiation factor 15 and NF-kappaB expression in benign prostatic biopsies and risk of subsequent prostate cancer detection. Cancer Med, 2021. 10(9): p. 3013-3025.
124. Gao, Y., et al., Growth differentiation factor-15 promotes immune escape of ovarian cancer via targeting CD44 in dendritic cells. Exp Cell Res, 2021. 402(1): p. 112522.
125. Yan, Y., et al., The expression of growth differentiation factor 15 in gallbladder carcinoma. J BUON, 2021. 26(1): p. 218-228.
126. Hegab, H.M., et al., Prognostic Impact of Serum Growth Differentiation Factor 15 Level in Acute Myeloid Leukemia Patients. Indian J Hematol Blood Transfus, 2021. 37(1): p. 37-44.
127. Deng, J., et al., Value of Growth/Differentiation Factor 15 in Diagnosis and the Evaluation of Chemotherapeutic Response in Lung Cancer. Clin Ther, 2021. 43(4): p. 747-759.
128. Xu, C., et al., Serum macrophage inhibitory cytokine-1 as a clinical marker for non-small cell lung cancer. J Cell Mol Med, 2021. 25(6): p. 3169-3172.
129. Joshi, J.P., et al., Growth differentiation factor 15 (GDF15)-mediated HER2 phosphorylation reduces trastuzumab sensitivity of HER2-overexpressing breast cancer cells. Biochem Pharmacol, 2011. 82(9): p. 1090-9.
130. Tsui, K.H., et al., Growth differentiation factor-15 upregulates interleukin-6 to promote tumorigenesis of prostate carcinoma PC-3 cells. J Mol Endocrinol, 2012. 49(2): p. 153-63.
131. Griner, S.E., J.P. Joshi, and R. Nahta, Growth differentiation factor 15 stimulates rapamycin-sensitive ovarian cancer cell growth and invasion. Biochem Pharmacol, 2013. 85(1): p. 46-58.
132. Zhou, Z., et al., Growth differentiation factor-15 suppresses maturation and function of dendritic cells and inhibits tumor-specific immune response. PLoS One, 2013. 8(11): p. e78618.
133. Rosenberg, S.A., J.C. Yang, and N.P. Restifo, Cancer immunotherapy: moving beyond current vaccines. Nat Med, 2004. 10(9): p. 909-15.
134. Hsu, F.J., et al., Tumor-specific idiotype vaccines in the treatment of patients with B-cell lymphoma--long-term results of a clinical trial. Blood, 1997. 89(9): p. 3129-35.
135. Neelapu, S.S., et al., Phase III randomized trial of patient-specific vaccination for previously untreated patients with follicular lymphoma in first complete remission: protocol summary and interim report. Clin Lymphoma, 2005. 6(1): p. 61-4.
136. Rosenberg, S.A., J.C. Yang, and N.P. Restifo, Reply-Cancer Vaccines: Pessimism in Check. Nat Med, 2004. 10(12): p. 1279-80.
137. Angelopoulou, K., et al., p53 gene mutation, tumor p53 protein overexpression, and serum p53 autoantibody generation in patients with breast cancer. Clin Biochem, 2000. 33(1): p. 53-62.
138. Disis, M.L., et al., Generation of T-cell immunity to the HER-2/neu protein after active immunization with HER-2/neu peptide-based vaccines. J Clin Oncol, 2002. 20(11): p. 2624-32.
139. Disis, M.L., et al., Generation of immunity to the HER-2/neu oncogenic protein in patients with breast and ovarian cancer using a peptide-based vaccine. Clin Cancer Res, 1999. 5(6): p. 1289-97.
140. Goydos, J.S., et al., A phase I trial of a synthetic mucin peptide vaccine. Induction of specific immune reactivity in patients with adenocarcinoma. J Surg Res, 1996. 63(1): p. 298-304.
141. Sandmaier, B.M., et al., Evidence of a cellular immune response against sialyl-Tn in breast and ovarian cancer patients after high-dose chemotherapy, stem cell rescue, and immunization with Theratope STn-KLH cancer vaccine. J Immunother, 1999. 22(1): p. 54-66.
142. Tilkin, A.F., et al., Primary proliferative T cell response to wild-type p53 protein in patients with breast cancer. Eur J Immunol, 1995. 25(6): p. 1765-9.
143. Jager, D., et al., Identification of tumor antigens as potential target antigens for immunotherapy by serological expression cloning. Cancer Immunol Immunother, 2004. 53(3): p. 144-7.
144. Ko, B.K., et al., Clinical studies of vaccines targeting breast cancer. Clin Cancer Res, 2003. 9(9): p. 3222-34.
145. Musselli, C., et al., Reevaluation of the cellular immune response in breast cancer patients vaccinated with MUC1. Int J Cancer, 2002. 97(5): p. 660-7.
146. Tsuboi, A., et al., Enhanced induction of human WT1-specific cytotoxic T lymphocytes with a 9-mer WT1 peptide modified at HLA-A *2402-binding residues. Cancer Immunol Immunother, 2002. 51(11-12): p. 614-20.
147. Gaffey, M.J., et al., Medullary carcinoma of the breast. Identification of lymphocyte subpopulations and their significance. Mod Pathol, 1993. 6(6): p. 721-8.
148. Harris, J.R., M. Marrow, and G. Bonadonna, Cancer of the Breast, in CANCER: Principles and Practice of Oncology, S.H. VT Devita, SA Rosenberg, Editor. 1993, J.B. Lippincott Co.: Philadelphia, PA.
149. Ridolfi, R.L., et al., Medullary carcinoma of the breast: a clinicopathologic study with 10 year follow-up. Cancer, 1977. 40(4): p. 1365-85.
150. Cohen, E.P. and T.S. Kim, Neoplastic cells that express low levels of MHC class I determinants escape host immunity. Semin Cancer Biol, 1994. 5(6): p. 419-28.
151. June, C.H., et al., The B7 and CD28 receptor families. Immunol Today, 1994. 15(7): p. 321-31.
152. Sinha, P., V.K. Clements, and S. Ostrand-Rosenberg, Interleukin-13-regulated M2 macrophages in combination with myeloid suppressor cells block immune surveillance against metastasis. Cancer Res, 2005. 65(24): p. 11743-51.
153. Agrawal, B., et al., Cancer-associated MUC1 mucin inhibits human T-cell proliferation, which is reversible by IL-2. Nat Med, 1998. 4(1): p. 43-9.
154. Hiltbold, E.M., et al., The mechanism of unresponsiveness to circulating tumor antigen MUC1 is a block in intracellular sorting and processing by dendritic cells. J Immunol, 2000. 165(7): p. 3730-41.
155. Smith-Thomas, L.C., J.P. Davis, and M.L. Epstein, The gut supports neurogenic differentiation of periocular mesenchyme, a chondrogenic neural crest-derived cell population. Dev Biol, 1986. 115(2): p. 293-300.
156. Vittoria, A., et al., VIP-immunoreactive nerve structures of the gastrointestinal tract in the developing and adult domestic duck. Arch Histol Cytol, 1992. 55(4): p. 361-74.
157. Zurn, A., Fibroblast growth factor differentially modulates the neurotransmitter phenotype of cultured sympathetic neurons. J Neurosci, 1992. 12(11): p. 4195-201.
158. Edyvane, K.A., et al., Regional differences in the innervation of the human ureterovesical junction by tyrosine hydroxylase-, vasoactive intestinal peptide- and neuropeptide Y-like immunoreactive nerves. J Urol, 1995. 154(1): p. 262-8.
159. Reubi, J.C., et al., Somatostatin and vasoactive intestinal peptide receptors in human mesenchymal tumors: in vitro identification. Cancer Res, 1996. 56(8): p. 1922-31.
160. Glazner, G.W., et al., Activity-dependent neurotrophic factor: a potent regulator of embryonic growth and development. Anat Embryol (Berl), 1999. 200(1): p. 65-71.

1. A method of preventing, treating, or reducing the risk of a disease, disorder, syndrome, symptom, and/or condition in an individual, comprising the steps of:
a. contacting a population of fibroblasts fibroblast-like cells; and/or extracellular vesicles with one or more hormones or hormone-like substances, thereby generating a contacted population; and
b. administering a therapeutically effective amount of the contacted population to the individual.

2. The method of claim 1, wherein the one or more hormones or hormone-like substances comprise one or more sex hormones.

3. The method of claim 1, wherein the one or more hormones or hormone-like substances are selected from the group consisting of testosterone, estrogen, adrenalin, melatonin, noradrenalin, norepinephrine, triiodothyronine (T3), thyroxine (T4), dopamine, prostaglandin-E2, prostaglandin-E1, leukotrienes, prostacyclin, thromboxane, amylin, anti-mullerian hormone, adiponectin, adrenocorticotropic hormone (ACTH), angiotensin, angiotensinogen, antidiuretic hormone (ADH), atrial natriuretic peptide (ANP), B-type natriuretic peptide (BNP), calcitonin, cholecystokinin (CCK), corticotrophin-releasing hormone (CRH), cortistatin, enkalphin, endothelin, estradiol, erythropoietin (EPO), follicle-stimulating hormone (FSH), galanin, gastric inhibitory peptide (GIP), gastrin, leukotriene, leptin, and a combination thereof.

4. The method of claim 1, wherein the disease is selected from the group consisting of depression, a wound, blindness, arthritis, ischemia, diabetes, endometriosis, multiple sclerosis, spinal cord injury, stroke, cancer, a lung disease, a blood disease, a neurological disease, an enzyme or hormone deficiency, a metabolic disorder, an autoimmune disease, age-related macular degeneration, retinal dystrophy, an infectious disease, hemophilia, a degenerative disease, an age-related disease, and a combination thereof.

5. The method of claim 4, wherein the arthritis is osteoarthritis or rheumatoid arthritis.

6. The method of claim 4, wherein the neurological disease is Parkinson’s disease, Alzheimer’s disease, Huntington’s disease, or amyotrophic lateral sclerosis.

7. The method of claim 4, wherein the metabolic disorder is a lysosomal storage disorder, galactosemia, maple syrup urine disease, phenylketonuria, a glycogen storage disease, a mitochondrial disorder, Friedrich’s ataxia, a peroxisomal disorder, a metal metabolism disorder, or an organic acidemia.

8. The method of claim 4, wherein the autoimmune disease is psoriasis, systemic lupus erythematosus, Grave’s disease, inflammatory bowel disease, Addison’s disease, Sjogren’s syndrome, Hashimoto’s thyroiditis, vasculitis, autoimmune hepatitis, alopecia areata, autoimmune pancreatitis, Crohn’s disease, ulcerative colitis, or dermatomyositis.

9. The method of claim 4, wherein the degenerative disease is Charcot-Marie-Tooth disease, chronic obstructive pulmonary disease, chronic traumatic encephalopathy, Creutzfeldt-Jakob disease, cystic fibrosis, cytochrome-C oxidase deficiency, Ehlers-Danlos syndrome, essential tremor, fribrodisplasia ossificans progressiva, infantile neuroaxonal dystrophy, keratoconus, keratoglobus, muscular dystrophy, neuronal ceroid lipofuscinosis, a prion disease, progressive supranuclear palsy, sandhoff disease, spinal muscular atrophy, or retinitis pigmentosa.

10. The method of claim 4, wherein the age-related disease is atherosclerosis, a cardiovascular disease, cataracts, osteoporosis, or hypertension.

11. The method of claim 10, wherein the cardiovascular disease is angina, or a myocardial infarction.

12. The method of claim 1, wherein the contacting and administering are done substantially simultaneously.

13. The method of claim 1, wherein the contacting comprises culturing the fibroblasts in the presence of the one or more hormones or hormone-like substances.

14. The method of claim 1, wherein the fibroblast population is allogeneic, autologous, or xenogeneic with respect to the individual.

15. The method of claim 1, wherein the fibroblast population comprises fibroblasts that are mitotically active.

16. The method of claim 1, wherein the fibroblast population is derived from tissue selected from the group consisting of skin, bone marrow, blood, mobilized peripheral blood, gingiva, tonsil, placenta, Wharton’s Jelly, hair follicle, fallopian tube, liver, deciduous tooth, vas deferens, endometrial, menstrual blood, omentum, and a combination thereof.

17. The method of claim 1, wherein the fibroblast population comprises fibroblasts that are plastic adherent, and/or wherein the fibroblast population is obtained by a purification method for the marker CD105.

18. (canceled)

19. The method of claim 17, wherein the purification method is flow cytometric purification and/or magnetic activated sorting.

20. The method of claim 1, wherein the fibroblast population comprises about 100,000 to about 300 million fibroblasts.

21. A method of preventing, treating, or reducing the risk of a disease or medical condition in an individual, comprising the step of administering to the individual one or more hormones and a population of fibroblasts.

22. The method of claim 21, wherein the disease or medical condition is endometriosis, depression, or a wound.

23. The method of claim 21 wherein the population of fibroblasts is contacted with the one or more hormones prior to the administering step.

24. The method of claim 23, wherein the population of fibroblasts is cultured with the one or more hormones.

25. The method of claim 22, wherein the disease is endometriosis and the hormone comprises estrogen.

26. The method of claim 25, wherein the estrogen is administered at a concentration of about 10 ng/kg of body weight to about 10 mg/kg of body weight, about 100 ng/kg of body weight to 1 about 0 mg/kg of body weight or about 100 ng/kg of body weight to 1 about 0 mg/kg of body weight.

27. (canceled)

28. (canceled)

29. The method of claim 25, wherein the fibroblast population comprises about 100,000 to about 300 million fibroblasts, about 100,000 to about 100 million fibroblasts, or about 100,000 to about 10 million fibroblasts.

30. (canceled)

31. (canceled)

32. The method of claim 22, wherein the disease is a wound and the hormone comprises leptin.

33. The method of claim 32, wherein the leptin and fibroblast population are administered at concentrations sufficient to accelerate wound healing and/or at concentrations sufficient to stimulate polarization of M1 macrophages to M2 macrophages.

34. (canceled)

35. The method of claim 33, wherein the leptin is administered at a concentration of about 1 pg/kg of body weight to about 100 µg/kg of body weight, about 100 ng/kg of body weight to about 100 µg/kg of body weight, about 100 ng/kg of body weight to about 10 µg/kg of body weight, or about 100 ng/kg of body weight to about 1 µg/kg of body weight.

36. (canceled)

37. (canceled)

38. (canceled)

39. The method of claim 33, wherein the fibroblast population comprises about 100,000 to about 300 million fibroblasts, about 100,000 to about 100 million fibroblasts, or about 100,000 to about 10 million fibroblasts.

40. (canceled)

41. (canceled)
